(19) United States
(12) Patent Application Publication
(10) Pub. No.: US 2022/0389414 A1
(43) Pub. Date: Dec. 8, 2022

(54) MOLECULAR COMPUTING METHODS AND SYSTEMS FOR SOLVING COMPUTATIONAL PROBLEMS

(71) Applicants: THE GOVERNING COUNCIL OF THE UNIVERSITY OF TORONTO, Toronto (CA); WISCONSIN ALUMNI RESEARCH FOUNDATION, Madison, WI (US); THE UNIVERSITY COURT OF THE UNIVERSITY OF GLASGOW, Glasgow (GB); PRESIDENT AND FELLOWS OF HARVARD COLLEGE, Cambridge, MA (US); NORTHWESTERN UNIVERSITY, Evanston, IL (US)

(72) Inventors: Alan ASPURU-GUZIK, Toronto (CA); Si Yue GUO, Toronto (CA); Tony Chang-Chi WU, Toronto (CA); Pascal Thomas FRIEDERICH, Karlsruhe (DE); Randall Howard GOLDSMITH, Madison, WI (US); Leroy CRONIN, Glasgow (GB); Abhishek SHARMA, Glasgow (GB); Yudong CAO, Cambridge, MA (US); Nathan C. GIANNESCHI, Wilmette, IL (US); Christopher James FORMAN, Chicago, IL (US)

(21) Appl. No.: 17/772,169

(22) PCT Filed: Oct. 28, 2020
(86) PCT No.: PCT/CA2020/051446

§ 371 Date: Apr. 27, 2022

Related U.S. Application Data
(60) Provisional application No. 62/926,971, filed on Oct. 28, 2019.

(51) Int. Cl. C12N 15/10; G06N 99/00; G06N 10/60

(52) U.S. Cl. CPC C12N 15/1089; G06N 99/007; G06N 10/60

(57) ABSTRACT

Molecular computer techniques for solving a computational problem using an array of reaction sites, for example, droplets, are disclosed. The problem may be represented as a Hamiltonian in terms of problem variables and problem parameters. The reaction sites may have a physicochemical property mapping to discrete site states corresponding to possible values of the problem variables. In a purely molecular approach, the reaction sites have intra-site and inter-site couplings enforced thereon representing the problem parameters, and the array is allowed to evolve, subjected to the enforced couplings, to a final configuration conveying a solution to the problem. In a hybrid classical-molecular approach, an iterative procedure may be performed that involves feeding read-out site states into a digital computer, determining, based on the problem parameters, perturbations to be applied to the states, and allowing the array to evolve under the perturbations to a final configuration conveying a solution to the problem.

### RELATED APPLICATIONS

The present application claims priority to U.S. Provisional Patent Application No. 62/926,971 filed on Oct. 28, 2019, the disclosure of which is incorporated herein by reference in its entirety.

### GOVERNMENT RIGHTS

This invention was made with government support under DARPA contract number W911NF-18-2-0036 awarded by the DARPA. The U.S. Government has certain rights in the invention.

### TECHNICAL FIELD

The general technical field relates to solving computational problems and, more particularly, to methods and systems for solving optimization problems based on molecular computing using, for example, microdroplet arrays.

### BACKGROUND

Maintaining the current pace at which science and technology are progressing is dependent on computational processing capabilities to keep growing. Conventional transistor-based computers, also referred to as classical computers, follow the von Neumann architecture, where information is processed in a central processing unit and stored in a memory unit. Different logic gates, each acting on a single bit or multiple bits of information represented as ones and zeros, are activated sequentially to perform an operation. However, as transistors are approaching tens of nanometers, corresponding to the size of large molecules, high heat dissipation and slow transfer rates between processors and memories are bringing about the breakdown of Moore's Law.

Alternatives to classical computers that are being developed include quantum computing, in which quantum mechanical phenomena allow information to exist as a superposition of states, rather than just as individual binary bits. For classical systems, computing with molecules is an attractive avenue given the vast chemical space and the relatively low energy dissipated in chemical reactions compared to the energy dissipated transistors. Proposals for computers that exploit chemical processes have taken either or both of two broad approaches: using chemistry and biochemistry to emulate circuit components or cellular automata; and employing a large number of molecules to explore a combinatorial space in parallel. Non-limiting examples of the first approach include reaction-diffusion systems, systems using the Belousov-Zhabotinsky (BZ) reaction, memristive polymers, transcription regulation for cellular signaling, and other chemical and biochemical analogs of logic gates. Non-limiting examples of the second approach include microfluidic devices, nanofabricated networks, and adaptive amoebal networks. DNA computers have explored both approaches, from early work that involved sifting through combinations of nucleic acid sequences to more recent work that have used DNA self-assembly to carry out an algorithm process. While each of these two approaches has certain advantages, each also has some drawbacks and limitations, including with respect to programmability, scaling, and maintaining accuracy in large calculations. Challenges therefore remain in the field of chemical and molecular computing.

### SUMMARY

The present disclosure generally relates to solving computational problems using molecular computing.

In accordance with an aspect, there is provided a molecular computer system for solving a computational problem represented as a problem Hamiltonian expressed in terms of a set of problem variables and a set of problem parameters, the molecular computer system including:
an array of reaction sites having a physicochemical property that maps to at least two possible site states, each reaction site representing one of the problem variables, the reaction sites having site couplings enforced thereon that represent the problem parameters,
wherein, in one or more runs, the array is allowed to evolve, starting from an initial array configuration and subjected to the enforced site couplings, toward a final array configuration in which each reaction site is in one of the at least two possible site states, the final array configuration providing a solution to the computational problem.

In some embodiments, the problem variables are binary variables, and the at least two possible site states of the physicochemical property are a pair of possible site states. In some embodiments, the problem variables are n-valued variables, and the at least two possible site states are a set of n possible site states, wherein n is a positive integer greater than two.

In some embodiments, the number of reaction sites is equal to the number of problem variables. In some embodiments, the number of reaction sites is greater than the number of problem variables.

In some embodiments, the site couplings include inter-site couplings. In some embodiments, the site couplings further include intra-site couplings.

In some embodiments, the molecular computer system further includes intra-site couplers and inter-site couplers configured to enforce the intra-site couplings and the inter-site couplings by mass exchange, respectively. In some embodiments, the inter-site couplers are configured to operate by passive diffusion or electrokinetic transport.

In some embodiments, the molecular computer system further includes intra-site couplers and inter-site couplers configured to enforce the intra-site couplings and the inter-site couplings by energy exchange, respectively. In some embodiments, the intra-site couplers and the inter-site couplers are part of an electrode array.

In some embodiments, the problem parameters include two-body interaction parameters encoded in the inter-site couplings. In some embodiments, the problem Hamiltonian is expressed in an Ising representation or a quadratic unconstrained binary optimization (QUBO) representation.

In some embodiments, the problem parameters include k-body interaction parameters encoded in the inter-site couplings, wherein k is a positive integer greater than two. In some embodiments, the problem Hamiltonian is expressed in a polynomial unconstrained binary optimization (PUBO) representation.

In some embodiments, the computational problem is a combinatorial optimization problem. In some embodiments, the combinatorial optimization problem is an NP or NP-hard optimization problem. In some embodiments, the NP or NP-hard optimization problem is an NP-complete optimization problem.

In some embodiments, the array of reaction sites includes an array of droplets. In some embodiments, the droplets each have a droplet volume ranging from about one nanoliter to about ten microliters.

In some embodiments, the array of reaction sites includes an array of containers, and the array of droplets is received in the array of containers.

In some embodiments, the array of reaction sites includes a non-wetting substrate, and the array of droplets is disposed on the non-wetting substrate.

In some embodiments, the array of reaction sites includes a gel substrate, and the array of droplets is embedded in the gel substrate.

In some embodiments, the array of reaction sites includes a printing substrate, and the array of droplets is printed on the printing substrate. In some embodiments, the printing substrate is made of paper or polymer.

In some embodiments, the array of reaction sites is arranged in a square lattice configuration or in a hexagonal lattice configuration.

In some embodiments, the physicochemical property includes pH, polymer molecular weight, concentration, oxidation state, color, viscosity, a chemical oscillation property, or a combination thereof.

In some embodiments, the molecular computer system further includes a site-readout device configured to read out the site states of the reaction sites in the final array configuration. In some embodiments, the site-readout device is configured to operate according to an optical readout scheme, an electrical readout scheme, an electrochemical readout scheme, or a combination thereof.

In some embodiments, the molecular computer system further includes a site-manipulation device configured to prepare the array of reaction sites in the initial array configuration. In some embodiments, the site-manipulation device is configured to operate according to an optical actuation scheme, an electrical actuation scheme, an electrochemical actuation scheme, or a combination thereof.

In some embodiments, each reaction site is in one of the at least two possible site states in the initial array configuration.

In some embodiments, the solution to the computational problem corresponds to a ground state of the problem Hamiltonian.

In some embodiments, the solution to the computational problem includes a plurality of solutions.

In accordance with another aspect, there is provided a molecular computer method for solving a computational problem represented as a problem Hamiltonian expressed in terms of a set of problem variables and a set of problem parameters, the molecular computer method including:
providing an array of reaction sites having a physicochemical property that maps to at least two possible site states, each reaction site representing one of the problem variables, the reaction sites having site couplings enforced thereon that represent the problem parameters;
performing, until an end condition has been met, one or more runs toward a solution to the computational problem, each run including:
   allowing the array to evolve, starting from an initial array configuration and subjected to the enforced site couplings, to a final array configuration in which each reaction site is in one of the at least two possible site states;
   reading out the site states of the reaction sites in the final array configuration;
   determining a candidate solution to the computational problem from the read-out site states; and
   determining whether the end condition has been met; and
determining the solution to the computational problem from at least one of the one or more candidate solutions.

In some embodiments, the problem variables are binary variables, and the at least two possible site states of the physicochemical property are a pair of possible site states. In some embodiments, the problem variables are n-valued variables, and the at least two possible site states are a set of n possible site states, wherein n is a positive integer greater than two.

In some embodiments, providing the array of reaction sites includes setting the number of reaction sites to be equal to the number of problem variables. In some embodiments, providing the array of reaction sites includes setting the number of reaction sites to be greater than the number of problem variables.

In some embodiments, the site couplings include inter-site couplings. In some embodiments, the site couplings further include intra-site couplings.

In some embodiments, providing the array of reaction sites includes enforcing the intra-site and the inter-site couplings by mass exchange. In some embodiments, providing the array of reaction sites includes enforcing the intra-site and the inter-site couplings by energy exchange.

In some embodiments, the problem parameters include two-body interaction parameters encoded in the inter-site couplings. In some embodiments, the problem Hamiltonian is expressed in an Ising representation or a quadratic unconstrained binary optimization (QUBO) representation.

In some embodiments, the problem parameters include k-body interaction parameters encoded in the inter-site couplings, wherein k is a positive integer greater than two. In some embodiments, the problem Hamiltonian is expressed in a polynomial unconstrained binary optimization (PUBO) representation. In some embodiments, the molecular computer method further includes performing a locality reduction process to reduce the k-body interaction parameters into two-body interaction parameters.

In some embodiments, the computational problem is a combinatorial optimization problem. In some embodiments, the combinatorial optimization problem is an NP or NP-hard optimization problem. In some embodiments, the NP or NP-hard optimization problem is an NP-complete optimization problem.

In some embodiments, the array of reaction sites includes an array of droplets. In some embodiments, providing the array of reaction sites includes disposing the array of droplets in an array of containers; or disposing the array of droplets on a non-wetting substrate; or embedding the array of droplets in a gel substrate; or printing the array of droplets on a printed substrate.

In some embodiments, the molecular computer method further includes selecting the physicochemical property from pH, polymer molecular weight, concentration, oxidation state, color, viscosity, a chemical oscillation property, or a combination thereof.

In some embodiments, reading out the site states of the reaction sites in the final array configuration includes operating an optical readout scheme, an electrical readout scheme, an electrochemical readout scheme, or a combination thereof.

In some embodiments, the molecular computer method further includes preparing the array of reaction sites in the initial array configuration. In some embodiments, preparing the array of reaction sites in the initial array configuration includes operating an optical actuation scheme, an electrical actuation scheme, an electrochemical actuation scheme, or a combination thereof.

In some embodiments, each reaction site is in one of the at least two possible site states in the initial array configuration.

In some embodiments, the one or more runs consist of a single run. In some embodiments, the one or more runs consist of multiple runs.

In some embodiments, the solution to the computational problem includes a plurality of solutions.

In some embodiments, determining whether the end condition has been met includes, for each run, assessing whether a specified number of run or runs has been completed, or assessing whether a specified allowed computation time has been reached, or assessing whether the candidate solution meets a specified solution criterion, or any combination thereof.

In some embodiments, the solution to the computational problem corresponds to a ground state of the problem Hamiltonian.

In accordance with another aspect, there is provided a hybrid classical-molecular computer (HCMC) system for solving a computational problem represented as a problem Hamiltonian expressed in terms of a set of problem variables and a set of problem parameters, the HCMC system including:
an array of reaction sites having a physicochemical property that maps to at least two possible site states, each reaction site representing one of the problem variables;
a digital computer operatively coupled to the array of reaction sites, the digital computer including a processor and a non-transitory computer readable storage medium having stored thereon computer readable instructions that, when executed by the processor, cause the processor to perform a method including:
   performing one or more iterative cycles toward a solution to the computational problem, each iterative cycle including:
      receiving, from a site-readout device, the site states of the reaction sites read out in an initial array configuration;
      determining a candidate solution to the computational problem from the initial array configuration by inputting the read-out site states for the problem variables in the problem Hamiltonian;
      determining whether an end condition has been met;
      if the end condition has been met, terminating the one or more iterative cycles; and
      if the end condition has not been met: determining, based on the problem parameters, state perturbations to be applied to a number of the reaction sites to promote state changes therein; controlling a site-manipulation device to apply the state perturbations; allowing the array to evolve, from the initial array configuration and responsive to the applied state perturbations, to a final array configuration; and
      setting the final array configuration as the initial array configuration for the next iterative cycle; and
   determining the solution to the computational problem from at least one of the one or more candidate solutions.

In some embodiments, the problem variables are binary variables, and the at least two possible site states of the physicochemical property are a pair of possible site states. In some embodiments, the problem variables are n-valued variables, and the at least two possible site states are a set of n possible site states, wherein n is a positive integer greater than two.

In some embodiments, the number of reaction sites is equal to the number of problem variables. In some embodiments, the number of reaction sites is greater than the number of problem variables.

In some embodiments, the problem parameters include two-body interaction parameters. In some embodiments, the problem Hamiltonian is expressed in an Ising representation or a quadratic unconstrained binary optimization (QUBO) representation.

In some embodiments, the problem parameters include k-body interaction parameters, wherein k is a positive integer greater than two. In some embodiments, the problem Hamiltonian is expressed in a polynomial unconstrained binary optimization (PUBO) representation.

In some embodiments, the computational problem is a combinatorial optimization problem. In some embodiments, the combinatorial optimization problem is an NP or NP-hard optimization problem. In some embodiments, the NP or NP-hard optimization problem is an NP-complete optimization problem.

In some embodiments, the array of reaction sites includes an array of droplets. In some embodiments, the droplets each have a droplet volume ranging from about one nanoliter to about ten microliters.

In some embodiments, the array of reaction sites includes an array of containers, and the array of droplets is received in the array of containers.

In some embodiments, the array of reaction sites includes a non-wetting substrate, and the array of droplets is disposed on the non-wetting substrate.

In some embodiments, the array of reaction sites includes a gel substrate and the array of droplets is embedded in the gel substrate.

In some embodiments, the array of reaction sites is arranged in a square lattice configuration or in a hexagonal lattice configuration.

In some embodiments, the physicochemical property includes pH, polymer molecular weight, concentration, oxidation state, color, viscosity, a chemical oscillation property, or a combination thereof.

In some embodiments, the site-readout device is configured to read out the site states of the reaction sites in the initial array configuration by operating an optical readout scheme, an electrical readout scheme, an electrochemical readout scheme, or a combination thereof.

In some embodiments, the site-manipulation device is configured to apply the state perturbations to the number of reaction sites by operating an optical actuation scheme, an electrical actuation scheme, an electrochemical actuation scheme, or a combination thereof.

In some embodiments, the solution to the computational problem corresponds to a ground state of the problem Hamiltonian.

In some embodiments, the solution to the computational problem includes a plurality of solutions.

In some embodiments, for each iterative cycle, the processor is configured for determining whether the end condition has been met by assessing whether a specified number of iterative cycles has been completed, or assessing whether a specified allowed computation time has been reached, or assessing whether the candidate solution meets a specified solution criterion, or any combination thereof.

In some embodiments, the processor is configured for determining the state perturbations by performing a computational optimization operation. In some embodiments, the computational optimization operation includes a simulated annealing operation or stochastic gradient descent operation.

In some embodiments, the processor is configured for: performing the one or more iterative cycles for a plurality of computational runs, each computational run having one or more candidate solutions associated therewith that define a respective one of a plurality of single-run solutions; and determining the solution to the computational problem from the plurality of single-run solutions.

In accordance with another aspect, there is provided a hybrid classical-molecular computer (HCMC) method for solving a computational problem represented as a problem Hamiltonian expressed in terms of a set of problem variables and a set of problem parameters, the hybrid classical-molecular method including:
providing an array of reaction sites having a physicochemical property that maps to at least two possible site states, each reaction site representing one of the problem variables;
performing one or more iterative cycles toward a solution to the computational problem, each iterative cycle including:
   reading out the site states of the reaction sites with the array in an initial array configuration;
   determining a candidate solution to the computational problem from the initial array configuration by inputting the read-out site states for the problem variables in the problem Hamiltonian;
   determining whether an end condition has been met;
   if the end condition has been met, terminating the one or more iterative cycles; and
   if the end condition has not been met:
      determining, based on the problem parameters, state perturbations to be applied to a number of the reaction sites to promote state changes therein;
      applying the state perturbations;
      allowing the array to evolve, from the initial array configuration and responsive to the applied state perturbations, to a final array configuration; and
      setting the final array configuration as the initial array configuration for the next iterative cycle; and
determining the solution to the computational problem from at least one of the one or more candidate solutions.

In some embodiments, the problem variables are binary variables, and wherein the at least two possible site states of the physicochemical property are a pair of possible site states. In some embodiments, the problem variables are n-valued variables, and wherein the at least two possible site states are a set of n possible site states, wherein n is a positive integer greater than two.

In some embodiments, providing the array of reaction sites includes setting the number of reaction sites to be equal to the number of problem variables. In some embodiments, providing the array of reaction sites includes setting the number of reaction sites to greater than the number of problem variables.

In some embodiments, the problem parameters include two-body interaction parameters. In some embodiments, the problem Hamiltonian is expressed in an Ising representation or a quadratic unconstrained binary optimization (QUBO) representation.

In some embodiments, the problem parameters include k-body interaction parameters, wherein k is a positive integer greater than two. In some embodiments, the problem Hamiltonian is expressed in a polynomial unconstrained binary optimization (PUBO) representation.

In some embodiments, the computational problem is a combinatorial optimization problem. In some embodiments, the combinatorial optimization problem is an NP or NP-hard optimization problem. In some embodiments, the NP or NP-hard optimization problem is an NP-complete optimization problem.

In some embodiments, the array of reaction sites includes an array of droplets. In some embodiments, providing the array of reaction sites includes disposing the array of droplets in an array of containers; or disposing the array of droplets on a non-wetting substrate; or embedding the array of droplets in a gel substrate; or printing the array of droplets on a printed substrate.

In some embodiments, the HCMC method further includes selecting the physicochemical property from pH, polymer molecular weight, concentration, oxidation state, color, viscosity, a chemical oscillation property, or a combination thereof.

In some embodiments, reading out the site states of the reaction sites in the initial array configuration including operating an optical readout scheme, an electrical readout scheme, an electrochemical readout scheme, or a combination thereof.

In some embodiments, applying the state perturbations to the number of reaction sites includes operating an optical actuation scheme, an electrical actuation scheme, an electrochemical actuation scheme, or a combination thereof.

In some embodiments, the solution to the computational problem corresponds to a ground state of the problem Hamiltonian.

In some embodiments, the solution to the computational problem includes a plurality of solutions.

In some embodiments, determining whether the end condition has been met includes, for each iterative cycle, assessing whether a specified number of iterative cycles has been completed, or assessing whether a specified allowed computation time has been reached, or assessing whether the candidate solution meets a specified solution criterion, or any combination thereof.

In some embodiments, determining the state perturbations includes performing a computational optimization operation. In some embodiments, the computational optimization operation includes a simulated annealing operation or stochastic gradient descent operation.

In some embodiments, the HCMC method further includes: performing the one or more iterative cycles for a plurality of computational runs, each computational run having one or more candidate solutions associated therewith that define a respective one of a plurality of single-run solutions; and determining the solution to the computational problem from the plurality of single-run solutions.

In accordance with another aspect, there is provided a non-transitory computer readable storage medium having stored thereon computer readable instructions that, when executed by a processor, cause the processor to perform a method for solving a computational problem using an array of reaction sites, the computational problem being represented as a problem Hamiltonian expressed in terms of a set of problem variables and a set of problem parameters, and the array of reaction sites having a physicochemical property that maps to at least two possible site states, each reaction site representing one of the problem variables, the method including:
performing, by the processor, one or more iterative cycles toward a solution to the computational problem, each iterative cycle including:
   receiving the site states of the reaction sites read out in an initial array configuration;
   determining a candidate solution to the computational problem from the initial array configuration by inputting the read-out site states for the problem variables in the problem Hamiltonian;
   determining whether an end condition has been met;
   if the end condition has been met, terminating the one or more iterative cycles; and
   if the end condition has not been met:
      determining, based on the problem parameters, state perturbations to be applied to a number of the reaction sites to promote state changes therein;
      controlling a site-manipulation device to apply the state perturbations;
      allowing the array to evolve, from the initial array configuration and responsive to the applied state perturbations, to a final array configuration; and
      setting the final array configuration as the initial array configuration for the next iterative cycle; and
determining, by the processor, the solution to the computational problem from at least one of the one or more candidate solutions.

In some embodiments, the problem variables are binary variables, and the at least two possible site states of the physicochemical property are a pair of possible site states.

In some embodiments, the problem parameters include two-body interaction parameters.

In some embodiments, the problem parameters include k-body interaction parameters, wherein k is a positive integer greater than two.

In some embodiments, the computational problem is a combinatorial optimization problem. In some embodiments, the combinatorial optimization problem is an NP or NP-hard optimization problem. In some embodiments, the NP or NP-hard optimization problem is an NP-complete optimization problem.

In some embodiments, the solution to the computational problem corresponds to a ground state of the problem Hamiltonian.

In some embodiments, the solution to the computational problem includes a plurality of solutions.

In some embodiments, determining whether the end condition has been met includes, for each iterative cycle, assessing whether a specified number of iterative cycles has been completed, or assessing whether a specified allowed computation time has been reached, or assessing whether the candidate solution meets a specified solution criterion, or any combination thereof.

In some embodiments, determining the state perturbations includes performing a computational optimization operation.

In some embodiments, the computational optimization operation includes a simulated annealing operation or stochastic gradient descent operation.

In some embodiments, the method further includes: performing the one or more iterative cycles for a plurality of computational runs, each computational run having one or more candidate solutions associated therewith that define a respective one of a plurality of single-run solutions; and determining the solution to the computational problem from the plurality of single-run solutions.

In accordance with another aspect, there is provided a digital computer including: a processor; and a non-transitory computer readable storage medium such as disclosed herein, the non-transitory computer readable storage medium being operatively coupled to the processor.

In accordance with another aspect, there is provided a hybrid classical-molecular computer (HCMC) system for solving a computational problem, the HCMC system including:
a droplet array including a set of droplets, each droplet having a controllable and measurable property mapping to a pair of droplet states; and
a digital computer operatively coupled to the droplet array, the digital computer including a processor and a non-transitory computer readable storage medium having stored thereon computer readable instructions that, when executed by the processor, cause the processor to perform a method including: (a) receiving the computational problem to be solved; (b) representing the computational problem to be solved as an Ising model, the Ising model including a set of interacting binary variables, each binary variable associated with a corresponding droplet, and a set of programmable model parameters encoding the computational problem to be solved as intra-droplet and inter-droplet interactions; (c) controlling a droplet-manipulation device to initialize the droplet array into an initial droplet state configuration; (d) controlling a droplet-measurement device to measure the set of droplet states; (e) using the set of measured droplet states into the Ising model to assess whether they provide an acceptable droplet state configuration; and (f) if yes, using the acceptable droplet state configuration as a solution to the computation problem; if no, determining an updated droplet state configuration, controlling the droplet-manipulation device to place the droplet array into the updated droplet state configuration, and repeating steps (d) to (f).

In accordance with another aspect, there is provided a hybrid classical-molecular computer (HCMC) method for solving a computational problem using a droplet array including a set of droplets, each droplet having a controllable and measurable property mapping to a pair of droplet states, the HCMC method including: (a) receiving the computational problem to be solved; (b) representing the computational problem to be solved as an Ising model, the Ising model including a set of interacting binary variables, each binary variable associated with a corresponding droplet, and a set of programmable model parameters encoding the computational problem to be solved as intra-droplet and inter-droplet interactions; (c) initializing the droplet array into an initial droplet state configuration; (d) measuring the set of droplet states; (e) using the set of measured droplet states into the Ising model to assess whether they provide an acceptable droplet state configuration; (f) if yes, using the acceptable droplet state configuration as a solution to the computation problem; if no, determining an updated droplet state configuration, controlling the droplet-manipulation device to place the droplet array into the updated droplet state configuration, and repeating steps (d) to (f).

In accordance with another aspect, there is provided a non-transitory computer readable storage medium having stored thereon computer executable instructions that, when executed by a processor, cause the processor to perform the HCMC method described herein.

In accordance with another aspect, there is provided a molecular computer system for solving a computational problem, the molecular system including a droplet array including a set of droplets, each droplet having a controllable and measurable property mapping to a pair of droplet states, the set of droplets having enforced intra-droplet and inter-droplet couplings acting thereon,
wherein the computational problem is represented as an Ising model including a set of interacting binary variables, each binary variable corresponding to a respective one of the droplets, and a set of model parameters defined by the enforced intra-droplet and inter-droplet couplings and encoding the computational problem to be solved, and
wherein, upon initializing the droplet array into an initial droplet state configuration, the droplet array is allowed, subjected to the intra-droplet and inter-droplet couplings acting thereon, to evolve thermodynamically to a steady-state droplet state configuration that corresponds to a solution of the computational problem.

In accordance with another aspect, there is provided a molecular computer method for solving a computational problem, the molecular computer method including: (a) providing a droplet array including a set of droplets, each droplet having a controllable and measurable property mapping to a pair of droplet states; (b) receiving the computational problem to be solved; (c) representing the computational problem to be solved as an Ising model, the Ising model including a set of interacting binary variables, each binary variable associated with a corresponding droplet, and a set of programmable model parameters encoding the computational problem to be solved; (d) enforcing intra-droplet and inter-droplet couplings to act on the set of droplets, the enforced intra-droplet and inter-droplet couplings representing the programmable model parameters of the Ising model; (e) initializing the droplet array into an initial droplet state configuration; (f) allowing the droplet array, subjected to the enforced intra-droplet and inter-droplet couplings acting thereon, to evolve thermodynamically to a steady-state droplet state configuration; (g) measuring the set of droplet states corresponding to a steady-state droplet state configuration; and (h) obtaining a solution to the computational problem based on the measured set of droplet states in the steady-state droplet state configuration.

It is appreciated that other method and process steps may be performed prior, during or after the steps described herein. The order of one or more steps may also differ, and some of the steps may be omitted, repeated, and/or combined, as the case may be. It is also to be noted that some steps may be performed using various analysis and processing techniques, which may be implemented in hardware, software, firmware, or any combination thereof.

Other objects, features, and advantages of the present description will become more apparent upon reading of the following non-restrictive description of specific embodiments thereof, given by way of example only with reference to the appended drawings. Although specific features described in the above summary and in the detailed description below may be described with respect to specific embodiments or aspects, it should be noted that these specific features may be combined with one another unless stated otherwise.

### BRIEF DESCRIPTION OF THE DRAWINGS

FIG. 1 is a flow diagram of a molecular computer method for solving a computational problem, in accordance with an embodiment.

FIG. 2 is a schematic representation of a molecular computer system for solving a computational problem, in accordance with an embodiment.

FIGS. 3A to 3D are schematic representations of molecular computer systems for solving a computational problem, in accordance with four different embodiments. In each of these embodiments, the molecular computer system includes an array of reaction sites that includes an array of droplets. In FIG. 3A, the array of reaction sites also includes an array of containers configured to receive the droplets therein. In FIG. 3B, the droplets are disposed on a non-wetting substrate. In FIG. 3C, the droplets are embedded in a gel substrate. In FIG. 3D, the array of reaction sites includes a printing substrate onto which the droplets are printed.

FIGS. 4A and 4B depict two examples of a physicochemical property of a droplet that can be mapped to two discrete droplet states to represent a binary variable of a problem Hamiltonian encoding a computational problem to be solved. In FIG. 4A, the physicochemical property is the pH of a droplet, and the two droplet states are acid (pH<7) and basic (pH>7). In FIG. 4B, the physicochemical property is the polymer molecular weight distribution a droplet, and the two droplet states are a low average molecular weight and high average molecular weight. It is noted that recent experiments have demonstrated the ability to optically monitor the degree of polymerization in microdroplets.

FIG. 5 is a schematic representation of a molecular computer system for solving a computational problem, in accordance with another embodiment.

FIGS. 6A and 6B are schematic representations of a molecular computer system including an array of reaction sites having intra-site and inter-site couplings enforced thereon that encode a computational problem to be solved by the molecular computer system. In FIG. 6A, the array of reaction sites is depicted in an initial array configuration. In FIG. 6B, the array of reaction sites is depicted in a final array configuration, to which it has been allowed to evolve from the initial array configured and subjected to the enforced intra-site and inter-site couplings.

FIG. 7 is a flow diagram of a hybrid-classical molecular computer method for solving a computational problem, in accordance with an embodiment.

FIG. 8 is a schematic representation of a hybrid-classical molecular computer system for solving a computational problem, in accordance with an embodiment.

FIGS. 9A to 9E relate to an example of a 2-SAT problem of 4 variables with 5 clauses. FIG. 9A depicts the clauses by lines connecting the droplets (solid line: “positive” coupling; dashed line: “negative” coupling). FIG. 9B depicts a random initialization of the problem, giving each droplet an initial value between −1 and +1. FIGS. 9C and 9D depicts the simulation of the steps of the optimization process. At each step, the droplet states are measured (FIG. 9C), and the gradients are calculated based on the Ising Hamiltonian (FIG. 9D). Once the system has reached a minimum, the final droplet states are mapped to a bit value of 0 or 1 (sᵢ∈[−1, 0)→xᵢ=0, sᵢ∈[0, 1]→xᵢ=1). FIG. 9E depicts a solution to the problem.

FIGS. 10A to 10D relate to an example of mapping bits to a hybrid and purely molecular computer for Boolean satisfiability problem of 9 variables with 17 clauses. FIG. 10A is a graph representing the problem (solid line: “positive” coupling; dashed line: “negative” coupling). FIGS. 10B to 10D depict a mapping of the problem to a droplet array for a hybrid classical-molecular computer (FIG. 10B), a purely molecular computer with a square lattice (FIG. 10C), and a purely molecular computer with a hexagonal lattice (FIG. 10D).

FIG. 11 depicts a graph of the probability of satisfiability as a function of clause density for a random 3-SAT problem obtained using a computational model of the HCMC approach disclosed herein (solid curves) and compared to solutions of the Davis-Putnam-Logemann-Loveland (DPLL) algorithm (dotted curves). Each point in the graph is the average of up to 10,000 3-SAT problems.

FIGS. 12A to 12E relate to an example of a lattice protein model simulated using a computational model of the HCMC approach disclosed herein. FIG. 12A illustrates the encoding of each bond in an amino-acid sequence as two bits. FIG. 12B illustrates an example of a five-amino-acid sequence with the corresponding sequence of bits. FIG. 12C is a graph depicting the results of an HCMC simulation performed for a six-amino-acid sequence. Each curve in FIG. 12C relates the energy of the chain (log scale) plotted as a function of the number of steps used the simulation for one instance of random initialization followed by annealing to a local minimum. The folded configuration corresponding to each curve is depicted. The ground state configuration has an energy of −6. FIG. 12D is a contour plot of the energy landscape between four configurations of the lattice protein. FIG. 12E is a graph of the energy of the system on a line between the unfolded state 0001010 and the folded state 0001011.

FIG. 13 is a schematic representation of a fluorescence microscope used for reading out droplet states in a study of a physical implementation of the HCMC approach disclosed herein.

FIG. 14 is a fluorescence image of a droplet array tested in the HCMC study.

FIG. 15 is a graph of the fluorescence signal measured at an electrode as the voltage at the electrode was scanned back and forth between 0 V and 5 V over many cycles, in the context of the HCMC study.

FIGS. 16A and 16B are fluorescence images of a droplet array having different voltages applied to the working electrodes (FIG. 16A: 0 V applied to all working electrodes; FIG. 16B: −5 V applied to all working electrodes), in the context of the HCMC study.

FIG. 17 is a graph of the time evolution of four droplet states of a droplet array during one run for solving a computational problem in the HCMC study.

FIG. 18 is a graph of the time evolution of the energy of the droplet array computed based on the droplet states depicted in FIG. 17.

FIG. 19A is a schematic representation of a model for a polymerization-based molecular computer implemented with a droplet array in which nearest-neighbor inter-droplet couplings are enforced by chemical mediator sites. FIG. 19B depicts the outcome of an HCMC-based simulation for solving a three-droplet 2-SAT problem using the model for a polymerization-based molecular computer of FIG. 19A.

FIG. 20A is a graph depicting five curves of the scaling of the number of droplets for solving five computational problems (lattice protein folding problem: two-local and four-local; Boolean satisfiability problem: 3-SAT, two-local and k-SAT, k-local), plotted as functions of the number of problem variables. FIG. 20B is a graph depicting fives curves of the number of problem variables that the five computational problems can include, plotted as functions of the number of droplets.

### DETAILED DESCRIPTION

In the present description, similar features in the drawings have been given similar reference numerals. To avoid cluttering certain figures, some elements may not be indicated if they were already identified in a preceding figure. It should also be understood that the elements of the drawings are not necessarily depicted to scale, since emphasis is placed on clearly illustrating the elements and structures of the present embodiments. Furthermore, positional descriptors indicating the location and/or orientation of one element with respect to another element are used herein for ease and clarity of description. Unless otherwise indicated, these positional descriptors should be taken in the context of the figures and should not be considered limiting. It is appreciated that such spatially relative terms are intended to encompass different orientations in the use or operation of the present embodiments, in addition to the orientations exemplified in the figures. Furthermore, when a first element is referred to as being “on”, “above”, “below”, “over”, or “under” a second element, the first element can be either directly or indirectly on, above, below, over, or under the second element, respectively, such that one or multiple intervening elements may be disposed between the first element and the second element.

The terms “a”, “an”, and “one” are defined herein to mean “at least one”, that is, these terms do not exclude a plural number of elements, unless stated otherwise.

The term “or” is defined herein to mean “and/or”, unless stated otherwise.

Terms such as “substantially”, “generally”, and “about”, which modify a value, condition, or characteristic of a feature of an exemplary embodiment, should be understood to mean that the value, condition, or characteristic is defined within tolerances that are acceptable for the proper operation of this exemplary embodiment for its intended application or that fall within an acceptable range of experimental error. In particular, the term “about” generally refers to a range of numbers that one skilled in the art would consider equivalent to the stated value (e.g., having the same or an equivalent function or result). In some instances, the term “about” means a variation of 10% of the stated value. It is noted that all numeric values used herein are assumed to be modified by the term “about”, unless stated otherwise. The term “between” as used herein to refer to a range of numbers or values defined by endpoints is intended to include both endpoints, unless stated otherwise.

The term “based on” as used herein is intended to mean “based at least in part on”, whether directly or indirectly, and to encompass both “based solely on” and “based partly on”. In particular, the term “based on” may also be understood as meaning “depending on”, “representative of”, “indicative of”, “associated with”, and the like.

The terms “match”, “matching”, and “matched” refer herein to a condition in which two elements are either the same or within some specified tolerance of each other. That is, these terms are meant to encompass not only “exactly” or “identically” matching the two elements but also “substantially”, “approximately”, or “subjectively” matching the two elements, as well as providing a higher or best match among a plurality of matching possibilities.

The terms “connected” and “coupled”, and derivatives and variants thereof, refer herein to any connection or coupling, either direct or indirect, between two or more elements, unless stated otherwise. For example, the connection or coupling between elements may be mechanical, optical, electrical, magnetic, thermal, chemical, logical, fluidic, operational, or any combination thereof.

The term “concurrently” refers herein to two or more processes that occur during coincident or overlapping time periods. The term “concurrently” does not necessarily imply complete synchronicity and encompasses various scenarios including: time-coincident or simultaneous occurrence of two processes; occurrence of a first process that both begins and ends during the duration of a second process; and occurrence of a first process that begins during the duration of a second process, but ends after the completion of the second process.

The terms “light” and “optical”, and variants and derivatives thereof, are intended to refer herein to radiation in any appropriate region of the electromagnetic spectrum. These terms are not limited to visible light but may also include invisible regions of the electromagnetic spectrum including, without limitation, radio, microwave (MW), terahertz (THz), infrared (IR), and ultraviolet (UV) spectral bands.

The present disclosure generally relates to methods and systems for solving optimization problems based on molecular computing. The search for novel or improved forms of computing as alternatives to traditional semiconductor-based computing following the von Neumann architecture is an active area of research. One such alternative is molecular computing, which is an interdisciplinary field that uses molecules and chemical reactions to perform calculations. The vast space of chemical reactions makes molecules a promising computational vehicle that may provide advantages in terms of reduced costs, room temperature operation, biocompatibility, energy efficiency, tunability, scalability, and inherent parallelization of processing and memory functions.

The present techniques perform computation using an array of spatially localized reaction sites, for example, an array of droplets, whose contents or properties represent bits of information. A computational problem to be solved is represented as a problem Hamiltonian expressed in terms of a set of problem variables and a set of problem parameters or constraints. The reaction sites have a physicochemical property, such as pH or polymer molecular weight, that maps to two or more possible site states, where each reaction site represents one of the problem variables.

In some embodiments, the present techniques make use of similarities between combinatorial optimization and statistical physics, notably the parallel that may be drawn between the cost function of an optimization problem and the energy of a physical system. Like the statistical ensemble behavior of physical systems such spin magnetic moments, an optimization problem may be thought of as a complex system of interacting variables. Such interactions may be mapped to a Hamiltonian or energy function, in which the constraints and requirements of the optimization problem correspond to interaction energies among and between the variables. For example, at a given temperature, the equilibrium distribution of an array of spin magnetic moments may follow a Boltzmann distribution, which favors the ground state of the Hamiltonian or, equivalently, the optimal solution or set of solutions to the objective function. Based on the fact that the statistical weight or probability of the ground state is inversely proportional to temperature, some embodiments of the present techniques may use simulated annealing to emulate the effect of this annealing process due to cooling for finding or approximating a ground state of a given problem Hamiltonian.

The present techniques disclose two main approaches to molecular computing, which are referred to herein as the purely molecular computer (PMC) approach and the hybrid classical molecular computer (HCMC) approach.

In the PMC approach, the reaction sites may have preprogrammed intra-site and inter-site couplings enforced thereon that represent the problem parameters of the problem Hamiltonian. The array thus prepared may be allowed, in one or more computational runs or tries, to evolve kinetically and/or thermodynamically and subjected to the enforced intra-site and inter-site couplings from an initial array configuration to a final array configuration. The final array configuration may correspond to a final state of the problem Hamiltonian, for example, a ground state, in which each reaction site is in one the possible site states. Reading out the site states of the reaction sites in the final array configuration may provide a solution to the computational problem.

In the HCMC approach, the site states of the reaction sites may be read out and the read-out states may be fed into a digital computer. The digital computer may be configured for inputting the read-out states for the problem variables in the problem Hamiltonian; performing calculations to determine, based on the problem parameters, state perturbations to be applied to a number of the reaction sites to promote site changes therein; applying the state perturbations to the number of reaction sites; and allowing the array to evolve, from an initial array configuration and as a result of the state perturbations, to a final array configuration which may be read out. One computational run in the HCMC approach may involve performing a number of iterations of these steps until an end condition is met. One or more computational runs may be performed to obtain a solution to the computation problem.

The present techniques have potential use in various industries and applications. Non-limiting examples of possible fields of use include, to name a few, logistics (e.g., industrial optimization, traffic/route optimization, supply chain optimization, job scheduling), data clustering, graph clustering, binary integer linear programming, finance (e.g. portfolio optimization), encryption (e.g., prime factoring), artificial intelligence and machine learning (e.g., Hopfield neural networks), software engineering, chemistry, pharmaceutical industry, and materials.

Various implementations of the present techniques will now be described with reference to the figures.

### Purely Molecular Computer Implementations

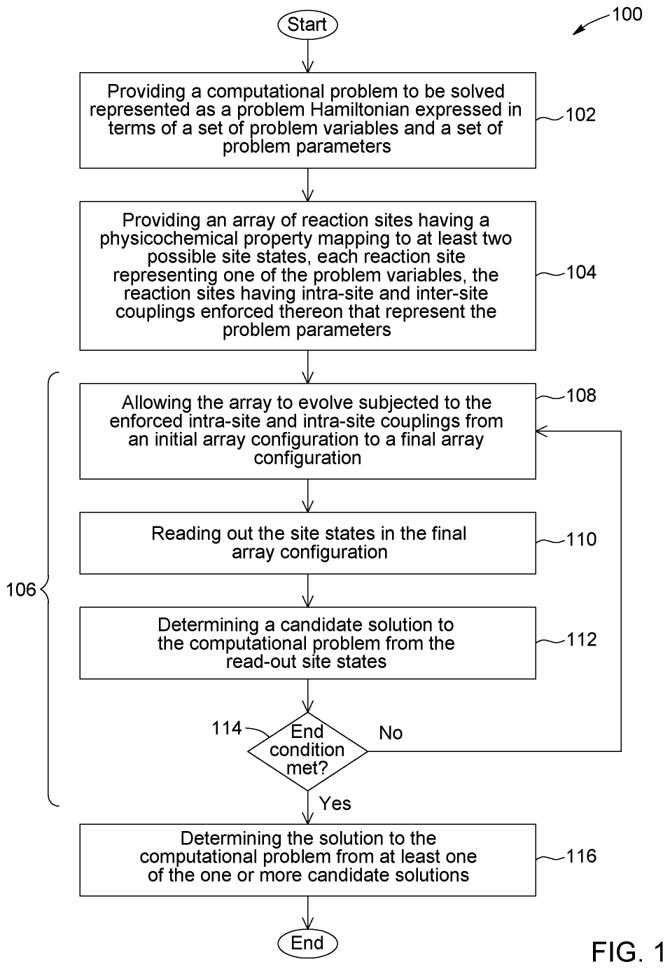

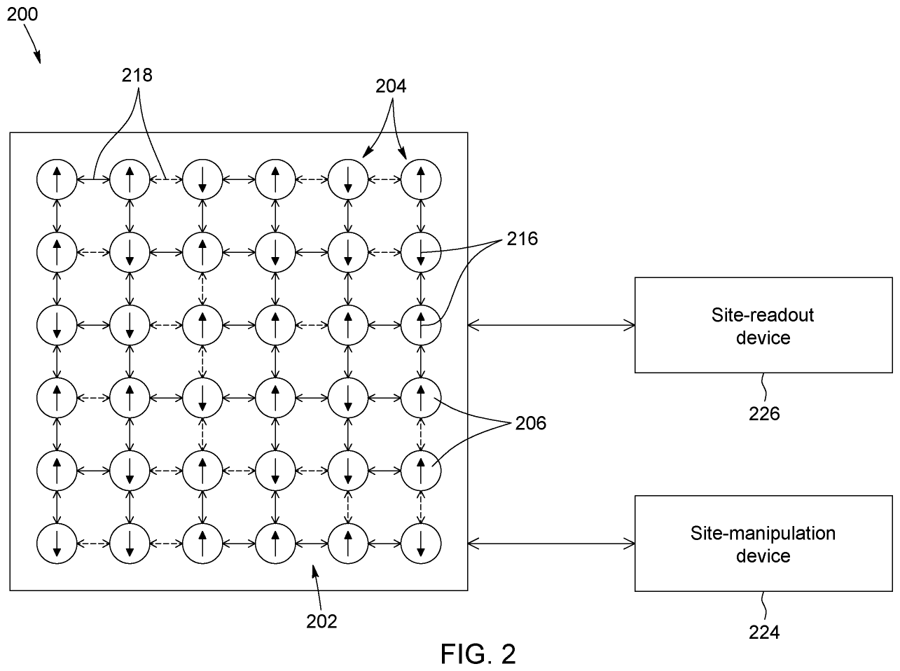

Referring to FIG. 1, there is provided a flow diagram of a PMC method 100 for solving a computational problem, in accordance with an embodiment. The method 100 of FIG. 1 may be implemented in a PMC system 200, such as the one depicted in FIG. 2, or another suitable PMC system.

The term “computational problem” broadly refers herein to any problem that might be solved by a computer. In some embodiments, the computational problem may be an optimization problem, such as a combinatorial optimization problem. Other possible types of computational problems include, for example, decision problems, search problems, and counting problems. The term “combinatorial optimization problem” refers herein to an optimization problem of finding an optimal solution from a finite set of solutions. In some embodiments, the combinatorial optimization problem may be a nondeterministic polynomial time (NP) or NP-hard optimization problem, for example, an NP-complete optimization problem. More specific examples of computational problems that may be solved using the present techniques include, without being limited to, the Boolean satisfiability (SAT) problem; the traveling salesperson person (TSP); the lattice protein folding problem; the prime factorization problem; the clique problem; the maximum cut problem; the knapsack problem; the subset sum problem; the number partitioning problem; the spin glass problem; the graph coloring problem; the graph similarity problem; the wedding table seating problem; the maximum independent set problem; the set cover problem; and the Hamiltonian cycle problem. It is appreciated that the theory and applications of computational problems, including optimization problems such as combinatorial optimization problem, are generally known in the art, and need not be described in detail herein other than to facilitate an understanding of the present techniques.

The PMC method 100 of FIG. 1 includes a step 102 of providing the computational problem as a problem Hamiltonian expressed in terms of a set of problem variables and a set of problem parameters.

The term “problem Hamiltonian” broadly refers herein to an energy, cost, or objective function that encodes the computational problem to be solved and that the method or system explores and attempts to sample from, minimize or maximize. It is appreciated that depending on the application and the computational problem to be solved, the problem Hamiltonian may take many different forms.

In some embodiments, the problem variables are binary variables, that is, variables which only can take two values, for example {0, 1} or {−1, 1}. It is appreciated that while in several embodiments described herein use problem Hamiltonians expressed in terms of binary problem variables, other embodiments may use problem Hamiltonians in which the problem variables are n-valued variables, where n is a positive integer greater than two. For example, the Potts model, which is a generalization of the Ising model, can have non-binary variables. It is also appreciated that techniques exist that can convert non-binary discrete variables into binary variables. Furthermore, the number of problem variables in the problem Hamiltonian can vary depending on the application. For example, in some embodiments, the number of problem variables may range between 10 and 2,000, although values outside this range are possible in other embodiments.

The problem parameters represent the constraints or requirements imposed by the computational problem on the problem variables. In some embodiments, the problem parameters include bias parameters and interaction parameters. In some applications, the interaction parameters include only two-body interactions between the problem variables, in which case the problem Hamiltonian can be said to be a two-local problem Hamiltonian. Non-limiting examples of possible two-local problem Hamiltonians are those expressed in an Ising formulation or a quadratic unconstrained binary optimization (QUBO) formulation. In other applications, the interaction parameters include k-body interaction parameters, k being a positive integer greater than two, in which case the problem Hamiltonian can be said to be a k-local problem Hamiltonian, for example, expressed in a polynomial unconstrained binary optimization (PUBO) formulation. It is appreciated that techniques exist that allow for a k-local problem Hamiltonian to be reframed as a two-local problem Hamiltonian, which may involve the use of ancillary variables.

A canonical model for spin magnetic moments arranged at lattice sites on a two-dimensional crystalline lattice is the Ising Hamiltonian:

$${H}_{P} = - μ h {\sum }_{i = 1}^{N} {s}_{i} - J {\sum }_{i < j}^{N} {s}_{i} {s}_{j} , {s}_{i} ∈ \{ - 1 , 1 \} \qquad ( 1 )$$

whose terms describe a tradeoff between the cost of flipping a spin sᵢ subjected to an external magnetic field h and the exchange energy J between neighboring spins, and where μ is the magnetic moment. Even though the Ising model has only two parameters, h and J, it may already give rise to computational problems that may become intractable in the worst cases. For encoding more general combinatorial optimization problems or allow for more degrees of freedom in defining the bias fields acting on the individual spins and the couplings between pairs of spins, a more general form of the Ising Hamiltonian may be written as follows:

$${H}_{P} = {\sum }_{i = 1}^{N} {α}_{i} {s}_{i} + {\sum }_{i < j}^{N} {β}_{ij} {s}_{i} {s}_{j} , \qquad ( 2 )$$

where the coefficients αᵢ and βᵢⱼ correspond to the problem parameters of the computational problem, with αᵢ representing the bias field acting on problem variable sᵢ, and βᵢⱼ representing the couplings between problems variables sᵢ and sⱼ. For given values of the vector α and the matrix β, the optimal configuration of sᵢ represents a solution that minimizes the problem Hamiltonian H_{P}. The problem Hamiltonian H_{P} of Eq. (2) may be generalized into a k-local problem Hamiltonian including higher-order interaction terms as follows:

$${H}_{P} = {h}^{( 0 )} + {\sum }_{i = 1}^{N} {h}_{i}^{( 1 )} {s}_{i} + {\sum }_{i < j}^{N} {h}_{i}^{( 2 )} {s}_{i} {s}_{j} + {\sum }_{i < j < k}^{N} {h}_{i}^{( 3 )} {s}_{i} {s}_{j} {s}_{k} + … , \qquad ( 3 )$$

where the term h^{(k)} is a rank-k tensor representing the coupling between k variables.

It is appreciated that implementing a problem Hamiltonian H_{P}, for example, an Ising Hamiltonian, in a programmable computer involves being able to control the bias fields and couplings for a set of problem variables. This approach to computation has been explored in other fields. In particular, the Ising model paradigm has inspired algorithm and hardware development for both classical and quantum devices. Classical implementations have been mostly based on traditional electronic components, such as a network of coupled LC oscillators, and conventional CMOS technologies, while quantum implementations have made use of quantum effects such as superposition and tunneling.

Quantum annealing and adiabatic quantum computing can provide a framework for solving computational problems using molecular computing. Quantum annealing is a metaheuristic approach to solving computational problems that use quantum mechanical effects, such as superposition, tunneling, and entanglement, to find a low-energy state, typically the ground state, of a system. Adiabatic quantum computing is a form of quantum annealing for which the system evolves under adiabatic conditions. In adiabatic quantum computing, a system is initially in the ground state of an initial Hamiltonian H₀ adiabatically evolves to a ground state of a final Hamiltonian H_{P}. The initial Hamiltonian H₀ typically encodes a known accessible computational problem, while the final Hamiltonian H_{P} is the problem Hamiltonian encoding the computational problem of interest. An example of an adiabatic evolution may be written as follows:

H(t)=[1−a(t)]H₀+a(t)H_{P}. (4)

To solve for the ground state of the problem Hamiltonian H_{P}, the instantaneous Hamiltonian H(t) is adiabatically changed over time from the initial Hamiltonian H₀ following a time-dependent function a(t). As the system evolves, the function a(t) goes from 0 to 1, such that the instantaneous Hamiltonian H(t) is equal to the initial Hamiltonian H₁ at the beginning of the evolution and is equal to the problem Hamiltonian H_{P} at the end of the evolution. In operation, the system is typically initialized in a ground state of the initial Hamiltonian H₀, and is then allowed to gradually evolve in a way such the system ends up in a ground state of the problem Hamiltonian H_{P} at the end of the evolution. If adiabatic conditions are not satisfied, the system may be excited to and end up in a higher-energy state, which is generally best avoided. It is appreciated that the theory and applications of quantum annealing and adiabatic quantum computing are generally known in the art, and need not be described in detail herein other than to facilitate an understanding of the present techniques.

Although a molecular computer remains a classical device, unable to avail itself of quantum effects, such as quantum tunneling across barriers in the potential energy surface associated with the problem Hamiltonian H_{P}, the rationale can still be extended to simulated annealing. Simulated annealing is a metaheuristic approach to solving computational problems by approximating the global optimum (or optima) of an objective function of a plurality of variables subjected to a set of constraints. Simulated annealing derives its name from annealing in metallurgy, as it models the physical process of heating a material and then gradually and controllably cooling the material to decrease defects in an attempt to minimize the system energy. In simulated annealing, H₀ represents the kinetic energy of the system, which may account for effects such as heat, noise, and diffusion, while H_{P} represents the potential energy of the system, which relates to the computational problem to be solved.

Simulated annealing was found to be well suited to tackle NP and NP-hard combinatorial optimization problems. These are problems whose computational complexity increases exponentially with the number of variables, and for which no polynomial-time algorithm is presently known. Nevertheless, since solving the Ising model was shown to be NP-hard, developing a good heuristic Ising model solver would be beneficial for solving real-world computational problems that are often NP-complete, such as the Boolean satisfiability problem, the traveling salesperson problem, and the graph coloring problem. As known in the art, NP-complete problems belong to both NP and NP-hard classes. They are defined as decision problems where a polynomial-time reduction exists from every other problem in NP. It was shown that that solving the states of the Ising model on a two-dimensional lattice with arbitrary interactions is NP-complete. Since every instance of a problem in NP can be efficiently encoded as an instance of an NP-complete problem, this implies that all problems in the NP complexity class may be mapped to instances of a two-dimensional Ising model, and thus may potentially be solved efficiently with a heuristic Ising solver. Table I presents the complexity of solving different Ising models.

TABLE I
Complexity of solving different Ising models.

| Dimension | Restrictions | Complexity solving |
|---|---|---|
| and graph | on coupling | for the Ising ground state |
| One-dimensional | None | Polynomial time (P) |
| Two-dimensional | hᵢ = 0 | P |
| (planar graph) | hᵢ ≠ 0 | NP-hard |
| Non-planar graph | Jᵢⱼ ∈{−1, 0, 1} | NP-hard |

It is appreciated that the theory and applications of simulated annealing are generally known in the art, and need not be described in detail herein other than to facilitate an understanding of the present techniques. It is also appreciated that while several embodiments disclosed herein use Ising and Ising-like problem Hamiltonians, various other types of problem Hamiltonians can be used in other embodiments.

Referring still to FIG. 1, the PMC method 100 also includes a step 104 of a providing an array of reaction sites having a physicochemical property that maps to at least two possible site states. The PMC system 200 of FIG. 2 includes an example of an array 202 of reaction sites 204. Each reaction site 204 represents one of the problem variables of the problem Hamiltonian encoding the computational problem to be solved. This means that if the problem variables are binary variables, then the possible site states of the physicochemical property are a pair of possible site states, whereas if the problem variables are n-valued variables, with n>2, then the possible site states are a set of n possible site states. More information regarding the physicochemical property associated with the reaction sites 204 is provided below.

The term “reaction site” refers herein to a localized region or area containing a substance or a reactant and in which a specified change or reaction involving the substance or reactant may occur. The change or reaction may modify or otherwise act on the physicochemical property mapping to the site state associated with the reaction site. The reaction site may include or be provided on a substrate or support structure where the substance or reactant may be disposed. In some embodiments, the reaction site includes a reaction chamber that at least partially encloses or otherwise defines a region or volume configured to receive the substance or reactant and compartmentalize the specified change or reaction. The reaction chamber may be separated from other such reaction chambers by walls or partitions. For example, the reaction chamber may include a cavity defined by the interior wall of a cell, a well, a receptacle, a reservoir, a compartment, a cartridge, a membrane, or any other suitable container. In other embodiments, the reaction site is isolated from other such reaction sites by spacing. For example, the spacing between neighboring reaction sites may be achieved by arranging the reaction sites in a spaced-apart configuration on a non-wetting substrate or inside a gel substrate. The reaction sites are also the locations at which the site states are read out.

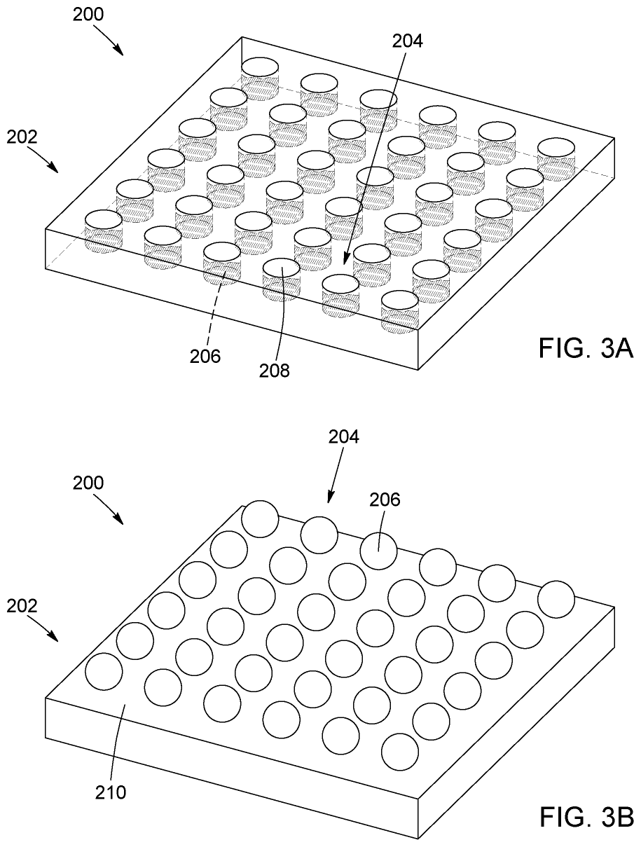

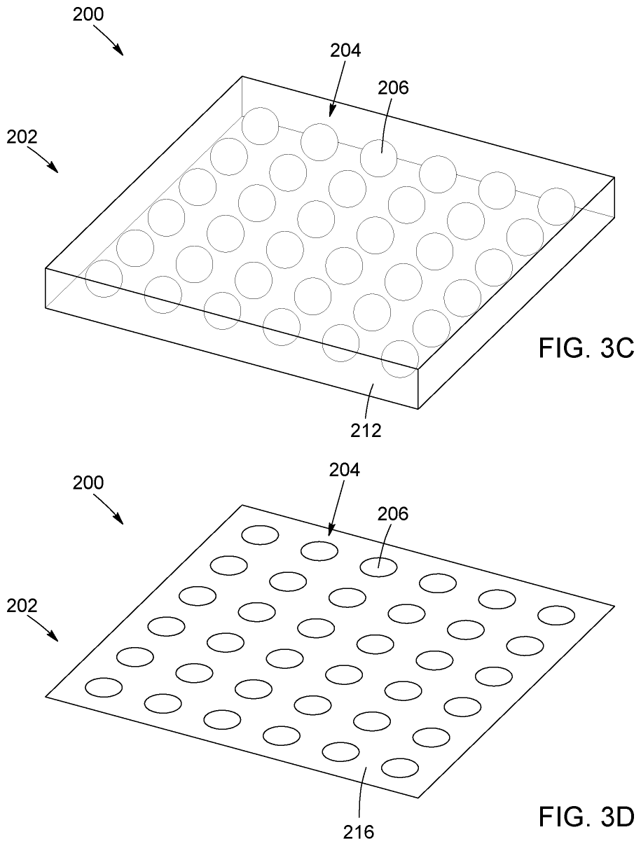

In some embodiments, the array 202 of reaction sites 204 includes an array of droplets 206. The term “droplet” refers herein to a discrete localized volume of liquid. Spatial confinement of droplets 206 can be achieved in various manners, non-limiting examples of which are depicted in FIGS. 3A to 3D. In each of these examples, the array 202 of reaction sites 204 includes an array of droplets 206. In FIG. 3A, the array 202 of reaction sites 204 includes an array of containers 208, for example, a microwell array or a microcell array. Each container 208 is associated with a respective one of the reaction sites 204 and is configured to receive therein a respective one of the droplets 206. In FIGS. 3B and 3C, spatial confinement of the array of droplets 206 is imposed by disposing the droplets 206 at discrete locations on a non-wetting substrate 210, where the droplets 206 may be held together by surface tension (FIG. 3B) and embedded in a gel substrate 212 (FIG. 3C). In FIG. 3D, the array 202 of reaction sites 204 includes a printing substrate 214, for example, a sheet of paper or made of another flexible material, such as a polymer, and the array of droplets 206 is printed on the printing substrate 214, for example, using an inkjet printer. Conventional inkjet printers are capable of printing droplets at different resolutions ranging, for example, from 150 dpi (dots per inch) to 1,200 dpi. It is appreciated that although FIGS. 3A to 3D illustrate embodiments where the droplets are spaced apart from one another (i.e., non-touching), other embodiments may include arrangements where droplets are in physical contact with their neighbors, including in a continuous phase.

The term “liquid” refers herein to a substance having a definite volume and the ability to flow and conforms to the shape of its container. The term “liquid” is meant to encompass fluid substances of various viscosities. Depending on the application, the term “liquid” may refer to a pure substance (e.g., water), a homogeneous solution containing one or more solutes dissolved in a solvent, a heterogeneous suspension, dispersion, emulsion, or multi-phase mixture, a cream, a gel, a paste, and the like. The terms “liquid” and “fluid” may generally be used interchangeably herein. The droplet may have various compositions. In particular, the droplet may include any liquid, including water, an aqueous solution, and oil solution, a polar liquid, a nonpolar liquid, polarizable liquid, and the like. The droplet may be of any suitable shape, whether spherical or nonspherical. The shape of a droplet may be altered by deposition onto a surface.

The droplet may be of any suitable size or volume. In one embodiment, the volume of a droplet may be less than one microliter, for example, between one nanoliter and one microliter, or between one nanoliter and ten microliters, or between one picoliter and one microliter, or between one picoliter and one nanoliter, including all values and ranges therebetween. In other embodiments, the volume of a droplet may be greater than one microliter. The term “microdroplet” may be used herein to refer to a droplet having a volume in the range from about one picoliter to about one microliter. It is appreciated, however, that the definition of a microdroplet may vary depending on the technical field under consideration, and is not meant to limit the scope of application of the embodiments disclosed herein. The terms “droplet” and “microdroplet” may generally be used interchangeably herein.

It is appreciated that droplets that may be prepared and arrayed using any suitable techniques. For example, microfluidic-based robotic platforms and inkjet printers can be used to generate droplets with precise compositions and to place the droplets at specified locations in an array. It is also appreciated that while several embodiments disclosed herein use reaction sites including droplets, other embodiments may not use droplets. For example, a continuous phase gel or liquid over an electrode array, where the reactions occur directly above the electrode.

Returning to FIG. 2, as noted above, the reaction sites 204 have a physicochemical property that maps to at least two possible site states. The possible site states associated with the physicochemical property are indicative of the possible values that can be taken by the problem variables of the problem Hamiltonian encoding the computational problem to be solved. The term “physicochemical property” is defined herein to broadly refer to any physical and/or chemical property of matter (e.g., a substance or reagent contained in the reaction sites 204) that may be defined, manipulated, read out, and mapped to two or more site states, so that each reaction site 204 may correspond to a discrete problem variable of a problem Hamiltonian encoding the computational problem to be solved. It is appreciated that while the measurements of physicochemical property often lie on a continuum, these measurements may be mapped or assigned to discrete site states using different techniques, for example, threshold-based techniques. Depending on the application, the physicochemical property may be a mechanical property, an electrical property, a chemical property, an electrochemical property, an optical property, a magnetic property, a thermal property, a fluidic property, an acoustic property, a biological property, or any combination thereof more specific examples of possible physicochemical properties that may be used in the embodiments disclosed herein include, without being limited to, pH; polymer molecular weight; concentration; oxidation state; color; viscosity; droplet size; absorption and emission spectra; fluorescence; phosphorescence; conductance; frequency, phase, amplitude, and other oscillating properties of chemical oscillators, such as the Belousov-Zhabotinsky (BZ) reaction and other autocatalytic reactions. When the reaction sites 204 contain droplets 206, the physicochemical property may be derived from a property of one or more of the droplet constituents. Conveniently, all the reaction sites may have the same physicochemical property, although it is not an absolute requirement.

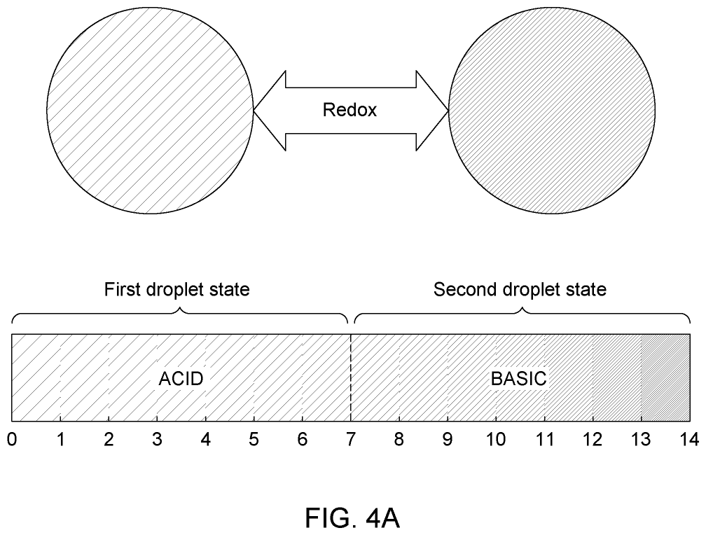

FIGS. 4A and 4B depict two examples of a physicochemical property of a droplet that can be mapped to two discrete droplet states to represent a binary variable of a problem Hamiltonian. In FIG. 4A, the physicochemical property is the pH of a droplet 206, and the two droplet states are acid, when the pH is less than 7, and basic, when the pH is greater than 7. The pH of a droplet may be reversibly and controllably changed using a variety of pH adjustment techniques. Non-limiting examples of such techniques include, to name a few, adding acid or base to the droplet, and applying electrical energy to the droplet to drive redox reactions that generate or consume H⁺. An implementation of the present techniques that uses pH as the physicochemical property is described below in the section entitled “Physical implementation of the HCMC approach”. In FIG. 4B, the physicochemical property is the polymer molecular weight distribution a droplet, which may be mapped to either a first droplet state, in which the polymer molecular weight distribution has a low average molecular weight (e.g., due to a high monomer concentration and/or a low polymer concentration), or a second droplet state, in which the polymer molecular weight distribution has a high average molecular weight (e.g., due to a low monomer concentration and/or a high polymer concentration). The polymer molecular weight distribution of a droplet may be controlled by polymerization and depolymerization processes, for example, using suitable catalysts. A model that uses polymer molecular weight distribution as the physicochemical property is described below in the section entitled “Model for a polymerization-based PMC approach”.

For illustrative purposes, the array 202 depicted in FIG. 2 includes 36 reaction sites 204 arranged in a 6×6 matrix. It is appreciated, however, that the number of reaction sites 204 in the array 202 may be higher or lower and may vary greatly depending on the application, notably on the computational problem to be solved. For example, in some embodiments, the array 202 can include from a few or a few tens of reaction sites 204 to thousands or tens of thousands of reaction sites 204. In other embodiments, the number of reaction sites 204 may be even greater, for example, in the order of millions or tens of millions. For example, about 30 million droplets could be printed at 600 dots per inch on a standard letter-size sheet of paper. Depending on the application, the number of reaction sites 204 may be equal to or greater than the number of problem variables. In the former case, each reaction site 204 represents a respective one of the problem variables, such that different reaction sites 204 correspond to different problem variables. In the latter case, each problem variable may be represented by one or more reaction sites 204, such that different reaction sites 204 may correspond to the same problem variable. More information regarding the number of reaction sites 204 and how it scales as a function of the number of problem variables is provided in the section entitled “Problem scaling” below.

In some embodiments, the reaction sites 204 are arranged in a two-dimensional lattice configuration. For example, in the embodiment of FIG. 2, the reaction sites are arranged in a square lattice configuration. In other embodiments, the reaction sites 204 are arranged in other lattice configurations including, but not limited to, rectangular, triangular, hexagonal, rhombic, parallelogrammic, pentagonal, and any other suitable regular or irregular lattice configurations. In yet other embodiments, the reaction sites 204 may be arranged in a linear array or be provided at arbitrary locations that do not conform to a specific pattern. In still further embodiments, the reaction sites 204 may be arranged in a three-dimensional array. Depending on the application, the spacing between neighboring reaction sites 204 may vary, for example, between a few micrometers to a few millimeters, although inter-site distances outside this range may be used in other applications. Furthermore, as noted above, reaction sites 204 may be touching in some applications.

The reaction sites 204 also have site couplings enforced thereon that represent the problem parameters. The site couplings may include inter-site couplings and inter-site couplings. In the PMC method 100 of FIG. 1, the step 104 of a providing the array 202 of reaction sites 204 includes a step of programming or encoding the problem parameters of the problem Hamiltonian into the intra-site couplings and inter-site couplings followed by a step enforcing the intra-site couplings and inter-site couplings on the reaction sites 204. The intra-site couplings and the inter-site couplings may be enforced by intra-site couplers 216 and inter-site couplers 218. It is appreciated that in addition to its the ability to be mapped onto a set of discrete and resolvable site states, the physicochemical property of a given reaction site 204 may also be reversibly changed between the different site states in accordance with the intra-site and inter-site couplings acting thereon. In particular, since the reaction sites 204 may be subjected to varying physical or chemical constraints from neighboring reaction sites 204 as the computation progresses, the physicochemical property of a given reaction site 204 may need to switch between its different site states several times over the course of one run of the PMC method 100.

The intra-site couplings represent bias fields that act on the physicochemical property of the individual reaction sites 204 to promote or favor one of their possible site states. That is, the intra-site coupling corresponding to a given reaction site 204 will not act on other reaction sites 204. For example, the intra-site coupling in each reaction site 204 may be programmed into the contents of the substance or reactant (e.g., the droplet) contained in the reaction site 204 or into a local field (e.g., an electrical bias applied by a dedicated electrode embodying the intra-site coupler 216) acting on the reaction site 204. In some embodiments, the intra-site couplings may be described as being positive (denoted by upward arrows in FIG. 2) or negative (denoted by downward arrows in FIG. 2) depending on the direction of bias fields associated thereto. It is appreciated that, in some implementations, the intra-site couplings acting on some or all of the reaction sites 204 may be zero.

The inter-site couplings represent the physical and/or chemical interactions between the reaction sites 204. This means that if the problem Hamiltonian is a two-local Hamiltonian, for example, having an Ising or a QUBO representation, then the inter-site couplings encode only two-body interaction parameters. Likewise, if the problem Hamiltonian is a k-local Hamiltonian, for example, having a PUBO representation, then the inter-site couplings encode up to k-body interaction parameters. In some embodiments, the inter-site couplings between reaction sites may be described as being positive (denoted solid double-ended arrows in FIG. 2) or negative (denoted by dashed double-ended arrows in FIG. 2). For example, the states of a pair of binary-state droplets may be denoted by s₁ and s₂, where sᵢ, s₂∈{0, 1}. In the case of a positive inter-droplet coupling, the interaction energy between the droplets is minimized when the two droplets are in the same state, either s₁=s₂=0 or s₁=s₂=1. This means that if the two droplets are initially in different states (i.e. s₁=0 and s₂=1 or s₁=1 and s₂=0), the positive coupling will promote or favor either one of the droplets to switch state. In the case of a negative inter-droplet coupling, the interaction energy between the droplets is minimized when the two droplets are in the different states, either s₁=0 and s₂=1 or s₁=1 and s₂=0. This means that if the two droplets are initially in the same state (i.e., s₁=s₂=0 or s₁=s₂=1), the negative coupling will promote or favor either one of the droplets to switch state. The symmetry in the positive or negative coupling between two droplets will generally be broken as interactions with other droplets are considered, leading to possible frustration as the various inter-droplet couplings compete with one another. As for the intra-site couplings, it is appreciated that, in some implementations, the inter-site couplings acting between some of the reaction sites may be zero.

It is appreciated that in a PMC system 200, each reaction site 204 may be coupled only to those reaction sites with which it can physically interact. Thus, in several embodiments, the PMC system 200 may be limited to two-body nearest-neighbor interactions, so that each reaction site 204 only establishes pairwise couplings with its nearest neighbors (e.g., four in the case of a square lattice and six in the case of a hexagonal lattice). To overcome these limitations related to the sparse connectivity and the reduced locality, minor-embedding techniques and locality reduction algorithms may be used. For example, locality reduction algorithms to reduce a k-local problem Hamiltonian to a two-local Hamiltonian may involve adding ancillary reaction sites to the PMC system 200. However, in other embodiments, the PMC system 200 may not be limited to two-body interactions and/or to nearest-neighbor interactions.

Depending on the application, and notably on the physicochemical property associated with the reaction sites 204, the intra-site and inter-site couplings may be implemented in various manners.

In some embodiments, the intra-site and inter-site couplings are mediated by mass exchange. Mass exchange relies on the movement and transport rates of molecules in and out and between the reaction sites, for example, using passive transport (e.g., passive diffusion, osmosis, reverse osmosis), active transport (e.g., hydrodynamic transport or electrokinetic transport such as electrophoresis and electro-osmosis), or any combination thereof. For example, in some implementations, the inter-site couplers 218 may be embodied by mediator sites programmed or otherwise configured to enforce the inter-site couplings between the reaction sites. The mediator sites may be arranged in an array interlaced with the reaction site array 202. In some embodiments, the mediators may have to be activated or programmed, for example, using an optical or electrical stimulation. The mediator sites may include chemical mediator sites configured to allow for the controlled exchange of chemical species between the reaction sites 204 in accordance with the sign and strength of the inter-site couplings enforced thereon. An example of an embodiment using chemical mediator sites programmed for the selective release of initiator and depolymerizer molecules between two droplets in response to their respective polymer molecular weight distributions is described below in the section entitled “Model for a polymerization-based PMC approach”.

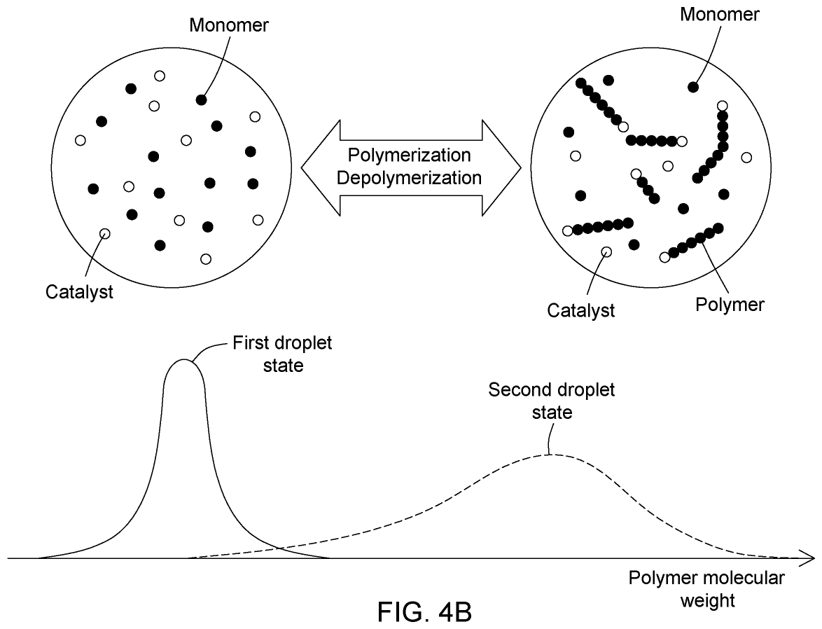

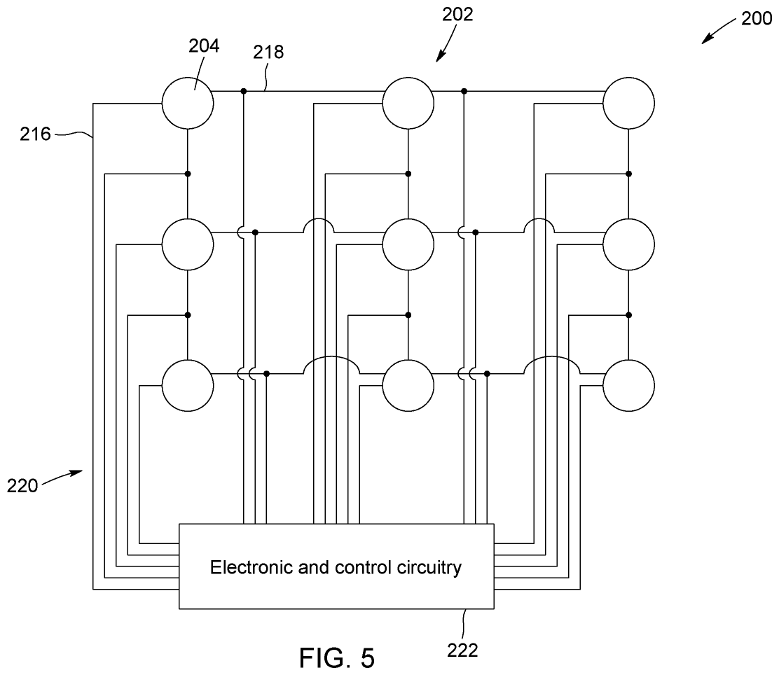

In other embodiments, the intra-site and inter-site couplings are mediated by energy exchange, for example, electrical energy transfer, electrochemical energy transfer, excitonic energy transfer, and Förster resonance energy transfer (FRET). For example, referring to FIG. 5, the PMC system 200 includes an array 202 of reaction sites 204 and an electrode array 220 with associated electronic and control circuitry 222. For example, the array 202 of reaction sites 204 may be disposed over the electrode array 220. The electrode array 220 may include a plurality of electrodes electrically connected to the reaction sites 204 and embodying the intra-site couplers 216 and the inter-site couplers 218. The intra-site and inter-site couplings representing the problem parameters of the problem Hamiltonian encoding the computational problem to be solved may be programmed into the electronic and control circuitry 222. The electronic and control circuitry 222 may be configured to control the plurality of electrodes embodying the intra-site couplers 216 and the inter-site couplers 218 to apply electrical signals to and between the reaction sites 204 to enforce the intra-site and inter-site couplings. In some embodiments, the electronic and control circuitry 222 may include a field-programmable gate array (FPGA) or another suitable computing device.

In other embodiments, the intra-site and inter-site couplings are mediated by both mass exchange and energy exchange.

Further non-limiting examples of implementing intra-site couplings include the following: for redox reactions, applying a constant voltage, for example, using a potentiostat; for certain types of polymerization like reversible addition-fragmentation chain transfer (RAFT), initiating the droplet array with different initiator or RAFT agent concentrations; for photoredox reactions, using a spatial light modulator (SLM) with a constant pattern to illuminate specific droplets; etching channels from a reservoir of a specific reaction to ensure constant supply.

Returning to FIG. 1, the PMC method 100 further includes a step 106 of performing, until an end condition has been met, one or more runs toward a solution to the computational problem.

The term “solution” refers herein to a parameter for which a suitable value or set of values is determined when an optimal value or set of values is sought. Depending on the application, the solution to the computational problem may be, but is not limited to, an optimal solution, an exact solution, an approximate solution, a solution meeting a solution criterion, a best feasible solution, a sufficiently good solution, or a solution providing a higher or best match among a plurality of candidate solutions. For example, in several cases, a sub-optimal solution that nevertheless fulfils one or more solution criteria may be considered to be an acceptable solution. The solution may include a plurality of solutions. In some embodiments, the solution may correspond to a global optimum (i.e., a global minimum or a global maximum) of the problem Hamiltonian encoding the computational problem to be solved, for example a ground state of the problem Hamiltonian. In some embodiments, the solution may correspond to an approximation of a global optimum, for example, an approximation of a ground state of the problem Hamiltonian. In some embodiments, the solution may correspond to a steady-state of the problem Hamiltonian. In some embodiments, the solution may include a plurality of solutions corresponding to a plurality of degenerate ground states of the problem Hamiltonian. In some embodiments, the solution may correspond to a local optimum (i.e., a local minimum or a local maximum). In some embodiments, the solution may be obtained from at least one of one or more candidate solutions obtained by performing the one or more runs.

Depending on the application, the term “end condition” may entail different things. In one example, the end condition may be reached after a specified or predetermined number of runs have been completed. In another example, the end condition may be reached after the expiration of a specified or predetermined time period. In yet another example, the end condition may be reached once a candidate solution meeting a specified or predetermined solution criterion has been obtained, for example, a candidate solution reaching a ground state.

Referring still to FIG. 1, each run may include a step 108 of allowing the array of reaction sites to evolve, starting from an initial array configuration and subjected to the enforced intra-site and intra-site couplings, to a final array configuration corresponding to a final state of the problem Hamiltonian and in which each reaction site is in one of the at least two possible site states. In some embodiments, the PMC method 100 includes a step of initializing the reaction site array in the initial array configuration. In such a case, the initial array configuration may be random, predetermined, or a combination thereof. The PMC system 200 may include a site-manipulation device 224 coupled to the reaction site array 202 and configured to set the initial array configuration. As discussed in greater detail below with respect to the HCMC approach, various types of site-manipulation devices 224 may be used, for example, based on optical, electrical and/or electrochemical actuation and control schemes. In this regard, it is appreciated that the site-manipulation device 224 may include or be connected to appropriate control and processing resources configured to control its operation. In other embodiments, however, the PMC method 100 may not include a specific step of initializing the reaction site array. In such a case, the initial array configuration may correspond to whichever configuration the array of reaction sites is into at the beginning of a given run.

In some embodiments, the initial array configuration may be characterized by having each reaction site in one of its possible site states. However, in other embodiments, the reaction sites may not be in one of their possible site states in the initial array configuration, though they may nevertheless end up being as such in the final array configuration. Such a scenario may arise when the physicochemical property mapping to the possible site states is not present in the reaction sites in the initial array configuration, but only emerges as the reaction proceeds. For example, in one embodiment, the physicochemical property may be the oscillating frequency of the BZ reaction, where a high value of oscillating frequency value may correspond to a first site state and a low value of oscillating frequency may correspond to a second site state. In such a case, the oscillations may start only after a perturbation is applied to the reaction site array, so that there may be no oscillations in the initial array configuration.

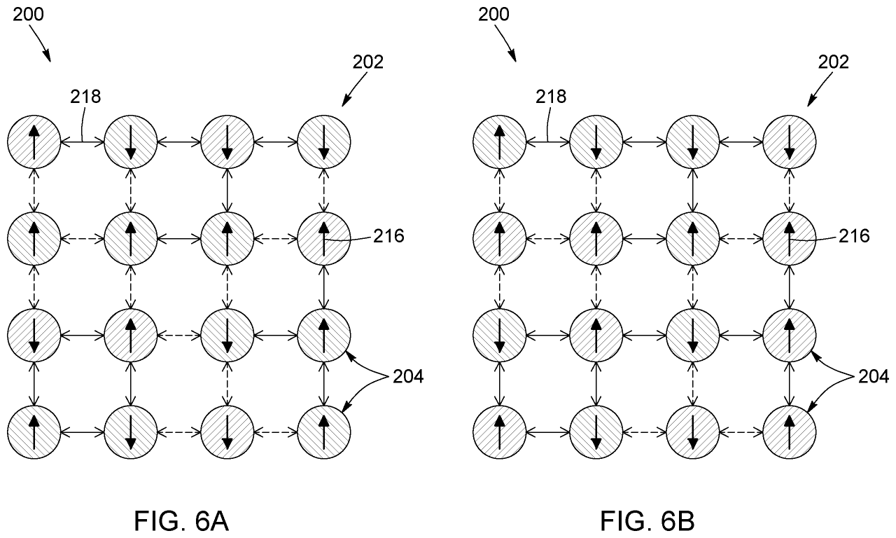

Referring to FIGS. 6A and 6B, there are illustrated schematic representations of a PMC system 200 including an array 202 of reaction sites 204 having intra-site couplings (up and down arrows) and inter-site couplings (solid and dashed doubled-ended arrows) respectively enforced thereon by intra-site couplers 216 and inter-site couplers 218. The intra-site and inter-site couplings represent the problem parameters of a problem Hamiltonian that encodes a computational problem to be solved by the PMC system 200. In FIG. 6A, the array 202 of reaction sites 204 is depicted in an initial array configuration. In FIG. 6B, the array 202 of reaction sites 204 is depicted in a final array configuration, to which it had evolved from the initial array configured and subjected to the intra-site and inter-site couplings respectively enforced by the intra-site couplers 216 and inter-site couplers 218. It is appreciated that between the initial array configuration in FIG. 6A and the final array configuration in FIG. 6B, each reaction site 204 has either remained in the same site state or evolved to the other site state. The two different states are represented by two different hatching orientations. It is also appreciated that the intra-site and inter-site couplings are the same in both the initial array configuration and the final array configuration.

It is emphasized that the PMC system 200 does not have a priori knowledge of the solution to the computational problem. Specifically, the PMC system 200 does not know beforehand the final array configuration it will end up in as the reaction site array 202 is allowed to evolve from a given initial array configuration. Rather, the PMC system 200 merely imposes, via the enforced intra-site and intra-site couplings representing the problem parameters of the problem Hamiltonian, the constraints of the computational problem to be solved on the reaction sites 204. As the reaction site array 202 evolves kinetically and/or thermodynamically under these conditions, it will attempt to minimize its free energy, which provides a physical impetus to explore distinct solution states associated with the problem Hamiltonian under the enforced intra-site and intra-site couplings. As noted above, depending on the nature and complexity of the computational problem, the problem Hamiltonian may have a single ground state corresponding to a single optimal final array configuration, a plurality of degenerate ground states corresponding to a plurality of optimal final array configurations, or one or more local minima that the PMC system identifies as “sufficiently good” solutions.

The amount of time taken by the PMC system 200 to evolve from an initial array configuration to a final, steady-state array configuration may vary depending on the application. In some embodiments, the amount of time needed from the PMC system 200 to go from the initial to the final array configuration may range from about a few seconds to about a few hours, although other embodiments may operate over different timescales.

Returning to FIG. 1, once the final array configuration has been reached, the PMC method 100 includes, for each run, a step 110 of reading out the site states of the reaction sites in the final array configuration. Referring to FIG. 2, the PMC system 200 includes a site-readout device 226 coupled to the reaction site array 202 and configured to read out the site states of the reaction sites 204.

It is appreciated that depending on the physicochemical property used, the site states of the reaction sites may be detected or measured—ideally in a noninterfering or negligibly interfering manner—using a variety of readout techniques including, but not limited to, an optical readout scheme, an electrical readout scheme, an electrochemical readout scheme, and any combination thereof. Non-limiting examples include, to name a few, fluorescence and spectroscopic measurements, fluorescence polarization measurements, color measurements (e.g., conveying information about oxidation states), electrical current measurements (e.g., in redox reactions), conductivity measurements, voltammetry, and the like. In one example, pH-sensitive dyes or ion indicators may be used to read out a fluorescence signal proportional to the pH or ion concentration of droplet species in the individual reaction sites. In another example, functionalized electrode arrays may be used to address the individual reaction sites and read out electrical signals conveying pH information about the reaction sites. In yet another example, fluorescent dyes sensitive to gelation or viscosity changes may be used to convey information about the presence of polymer. It is appreciated that various techniques are known in the art to detect, measure or otherwise read out droplet properties, which may be used in the embodiments herein.

The readout step 110 may include a step of processing or analyzing the measurements of the physicochemical property of the reaction sites in order to map the measurements to the possible site states. As noted above, because measurements of the physicochemical property can generally lie anywhere on a continuum of possible values, converting the array of measured data associated with the array of reaction sites into an array of read-out site states may involve some processing. To this end, the site-readout device 226 of the PMC system 200 in FIG. 2 may include or be connected to appropriate control and processing resources configured to determine read-out site states based on measured data conveying information about the physicochemical property of the reaction sites 204.

It is appreciated that depending on the physicochemical property used to provide the site states associated with the problem variables of the problem Hamiltonian encoding the computational problem to be solved, the evolution of the PMC system 200 from the initial to the final array configuration and its readout in the final array configuration may or may not be performed under controlled operating conditions. In particular, by selection of a suitable physicochemical property (e.g., some polymerization, redox, diffusion, and gelation reactions), some embodiments of the PMC method may be performed at or near room temperature, which may provide advantages over quantum computation techniques operating at cryogenic temperatures.

Returning to FIG. 1, each run further includes a step 112 of determining a candidate solution to the computational problem from the read-out site states, followed by a step 114 of determining whether the end condition has been met. In some embodiments, the set of read-out site states—one per reaction site—may directly provide the candidate solution to the computational problem associated with a given run. However, in other embodiments, the read-out site states may need to be processed in order to yield the candidate solution. As noted above, the step 114 of determining whether the end condition has been met may include, but is not limited to, any, some, or all of the following steps: assessing whether a specified number of runs has been completed; assessing whether a specified allowed computation time has been reached; and assessing whether the candidate solution meets a specified solution criterion. Non-limiting examples of solution criterion that may be applied at the i^{th} run include, to a name a few, H_{P,i}=0; H_{P,i}>H_{threshold} or H_{P,i}<H_{threshold}; |H_{P,i}−H_{P,i-1}|=0; |H_{P,i}−H_{P,i-1}|<ΔH_{threshold}; min{H_{P,1}, . . . , H_{P,i}}=H_{P,i}, or max{H_{P,1}, . . . , H_{P,i}}=H_{P,i}, and the like, where H_{P,i} is the value of the problem Hamiltonian corresponding to the i^{th} candidate solution, and H_{threshold} and ΔH_{threshold} are specified thresholds.

In some embodiments, only one run is performed so that only one candidate solution is determination. In other embodiments, a plurality of runs are performed, for example, ranging from two to about a hundred, resulting a plurality of candidate solutions being determined. It is appreciated that because of factors such as the shape of the potential energy surface associated with the problem Hamiltonian, the unavailability of quantum tunneling to pass through energy barriers, the reaction kinetics involved in the evolution of the reaction site array from the initial to the final array configuration, and the generally unavoidable presence of noise and experimental imperfections, a given run of the PMC method 100 may end up trapped in a local optimum of the solution space. In such a case, multiple runs may be required to find the global optimum (or optima). In particular, it is appreciated that depending on the shape of the potential energy surface associated with the problem Hamiltonian, the entrance to the ground state valley may be narrow and not readily accessible.

Referring still to FIG. 1, if the PMC method 100 determines that the end condition has not been met, then the method 100 performs another run, including the above-described allowing step 108, the readout step 110, and the determining steps 112, 114. However, if the PMC method 100 determines that the end condition has been met, the method 100 includes a step 116 of determining the solution to the computational problem from at least one of the one or more candidate solutions. It is appreciated that if only one candidate solution is determined, corresponding to only one run having been performed at step 106, then the solution to the computational problem is determined from the single candidate solution. However, if multiple candidate solutions have been determined, corresponding to multiple runs having been performed at step 106, then the solution to the computational problem is determined from at least one of the multiple candidate solutions. In some embodiments, the solution to the computational problem may correspond to a particular one of the multiple candidate solutions. In one example, the particular candidate solution may correspond to a ground state or a suitable approximation of a ground state of the problem Hamiltonian encoding the computational problem. In another example, the particular candidate solution may correspond to a best feasible solution to the computational problem. In yet another example, the particular candidate solution may provide a higher or best match among the multiple candidate solutions or a subset of the multiple candidate solutions. In other embodiments, the solution to the computational problem may correspond to a subset of the multiple candidate solutions, for example, if the problem Hamiltonian encoding the computational problem has a subset of degenerate ground states.

It is appreciated that some applications of the PMC approach may benefit from a complete parallelization of the problem solving process. For example, in the case of two-local problem Hamiltonian, a classical computer would generally perform, at each step of the optimization process, the computation of n² terms representing the pairwise energies of the n variables. In contrast, the PMC approach may perform the same computation n times faster since all pairwise interactions occur simultaneously, in O(n) time.

### Hybrid-Classical Molecular Computer Implementations

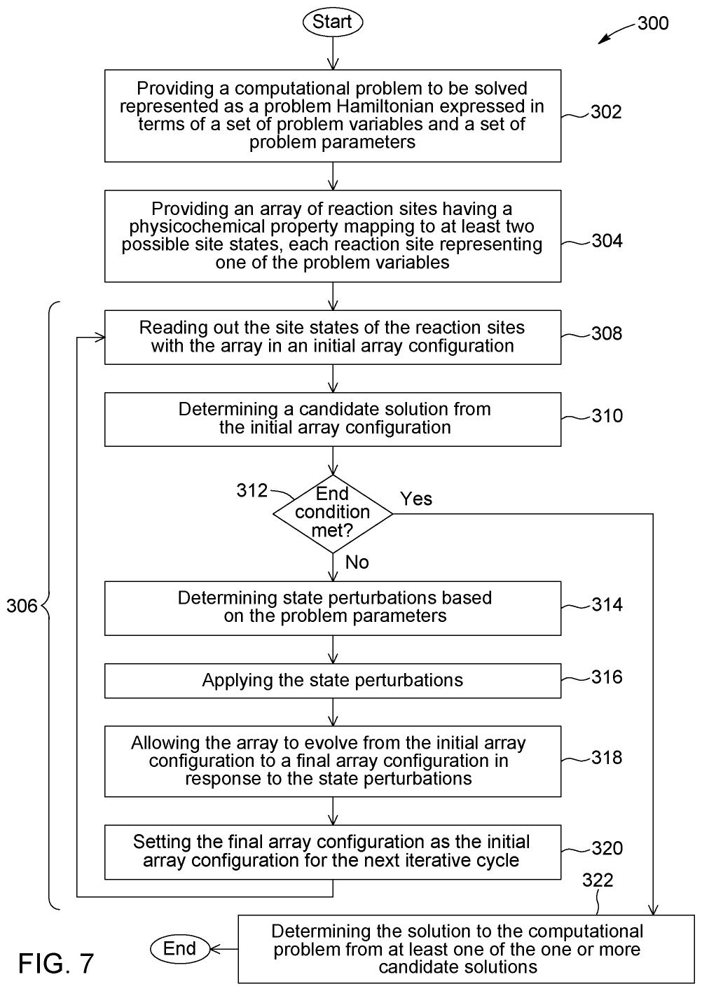

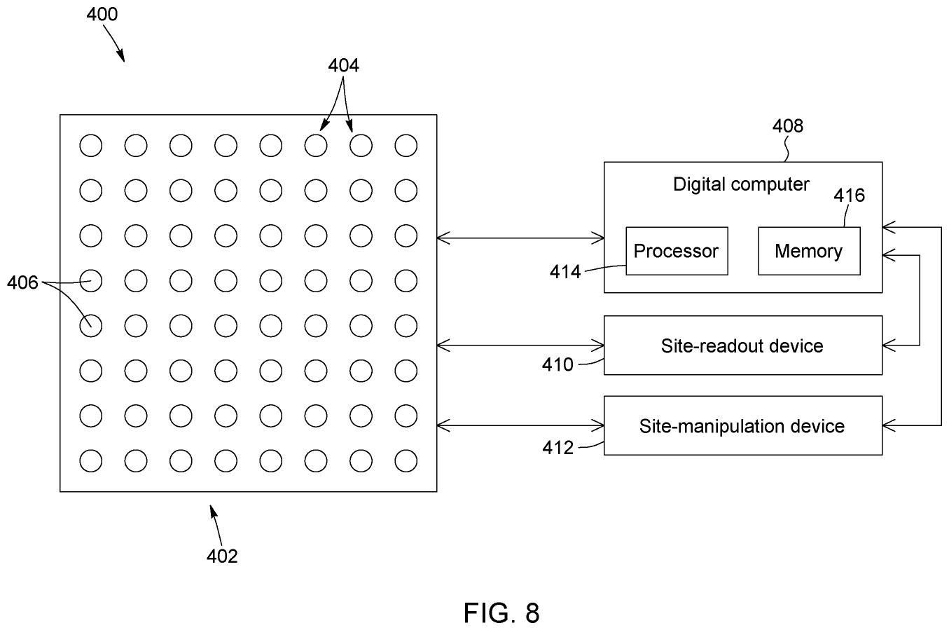

Referring now to FIG. 7, there is provided a flow diagram of a HCMC method 300 for solving a computational problem, in accordance with an embodiment. The method 300 of FIG. 7 may be implemented in a HCMC system 400, such as the one depicted in FIG. 8, or another suitable HCMC system. It is appreciated that the embodiments of FIGS. 7 and 8 may share several features with the embodiments described above with respect to the PMC approach. These shared features will not be described again in detail other than to highlight differences between them.

In some embodiments, the computational problem solved by the HCMC method 300 may be an optimization problem, such as a combinatorial optimization problem. In some embodiments, the combinatorial optimization problem may be an NP or NP-hard optimization problem, for example, an NP-complete optimization problem. More specific examples of computational problems that may be solved using the HCMC method 300 may include those listed above with respect to the PMC approach.

The HCMC method 300 includes a step 302 of providing the computational problem as a problem Hamiltonian expressed in terms of a set of problem variables and a set of problem parameters. The problem Hamiltonian used in the HCMC method 300 may take many different forms depending on the application. For example, depending on the computational problem to be solved, the problem variables may be binary variables or n-valued variables, where n>2. Furthermore, the number of problem variables in the problem Hamiltonian may vary depending on the application, as noted regarding the PMC approach. The problem parameters represent the constraints or requirements imposed by the computational problem on the problem variables. In some embodiments, the problem parameters may include bias parameters and interaction parameters. In some applications, the interaction parameters may include only two-body interactions between the problem variables, in which case the problem Hamiltonian can be said to be a two-local problem Hamiltonian (e.g., having an Ising or a QUBO representation). In other applications, the interaction parameters may include k-body interaction parameters, k≥2, in which case the problem Hamiltonian can be said to be a k-local problem Hamiltonian (e.g., having a PUBO representation). As noted above, locality reduction techniques exist for converting a k-local problem Hamiltonian into a two-local problem Hamiltonian.

Referring still to FIG. 7, the HCMC method 300 also includes a step 304 of providing an array of reaction sites having a physicochemical property that maps to at least two possible site states. The HCMC system 400 of FIG. 8 includes an example of an array 402 of reaction sites 404. Each reaction site 404 represents one of the problem variables of the problem Hamiltonian encoding the computational problem to be solved. This means that if the problem variables are binary variables, then the possible site states of the physicochemical property are a pair of possible site states, whereas if the problem variables are n-valued variables, with n>2, then the possible site states are a set of n possible site states.

The reaction sites 404 in the HCMC approach may share several features with those described above with respect to the PMC approach. The reaction sites 404 may include or be provided on a substrate or support structure where the substance or reactant may be disposed. In some embodiments, the reaction site may include a reaction chamber (e.g., a container, a cell, a well, a receptacle, a reservoir, a compartment, a cartridge) that at least partially encloses or otherwise defines a region or volume configured to receive a droplet or another object that may undergo a specified change or reaction. The reaction sites 404 may be separated from one another by physical barriers or spacing. In some embodiments, the array 402 of reaction sites 404 may include an array of droplets 406 arranged on a microwell array or on or in a substrate. The discussion above regarding various aspects and features relating to the use of droplets in the PMC approach generally applies to the HCMC approach.

Returning to FIG. 8, as noted above, the reaction sites 404 have a physicochemical property that maps to at least two possible site states. The possible site states associated with the physicochemical property are indicative of the possible values that can be taken by the problem variables of the problem Hamiltonian encoding the computational problem to be solved. The discussion above regarding various aspects and features relating to the physicochemical property in the PMC approach generally applies to the HCMC approach. In particular, depending on the application, the physicochemical property may be a mechanical property, an electrical property, a chemical property, an electrochemical property, an optical property, a magnetic property, a thermal property, a fluidic property, an acoustic property, a biological property, or any combination thereof. Furthermore, depending on the selected physicochemical property, the HCMC approach may or may not be performed under controlled operating conditions (e.g., in terms of temperature and/or pressure).

For illustrative purposes, the array 402 depicted in FIG. 2 includes 64 reaction sites 404 arranged in an 8×8 square lattice. It is appreciated, however, that the number of reaction sites 404 may be higher or lower and may vary greatly depending on the application, notably on the computational problem to be solved, as noted above with to the PMC approach. Depending on the application, the number of reaction sites 404 may be equal to or greater than the number of problem variables. In the former case, each reaction site 404 represents a respective one of the problem variables, such that different reaction sites 404 correspond to different problem variables. In the latter case, each problem variable may be represented by one or more reaction sites 404, such that different reaction sites 404 may correspond to the same problem variable. It is also appreciated that the spatial arrangement of the reaction sites 404 in the array 402 may be adjusted depending on the application.

In contrast to the PMC approach, the reaction sites 404 in FIG. 8 do not have intra-site couplings and inter-site couplings directly or physically enforced thereon, whose nature and strength represent the problem parameters. That is, in the HCMC system 400 of FIG. 8, the reaction sites 404 generally do not have bias fields applied thereto (i.e., other than state perturbations; see below) and generally do no interact physically or chemically with one another. Rather, the HCMC system 400 includes a digital computer 408 operatively coupled to the array 402 of reaction sites 404 and configured to enforce the constraints or requirements imposed by the computational problem. The term “digital computer” as used herein to refer to any classical computing or information processing system that follow the von Neumann architecture, where digital data is processed in a processor and stored in a memory. More specifically, as described in greater detail below, the digital computer 408 is configured, inter alia, for receiving read-out site states of the reaction sites 404 in an initial array configuration; computing, based on the read-out site states and the problem parameters, state perturbations to be applied to the reaction sites 404 to promote changes therein; and delivering instructions for applying the state perturbations to the reaction sites 404 to allow the reaction sites 404 to evolve to a final array configuration, which can read out and assessed as a candidate solution to the computation problem. In addition to the digital computer 408, the HCMC system 400 may further include a site-readout device 410 and a site-manipulation device 412, both of which coupled between the reaction site array 402 and the digital computer 408. The site-readout device 410 may be configured, inter alia, to read out the site states of the reaction sites 404, while the site-manipulation device 412 may be configured, inter alia, to initialize, vary, or otherwise manipulate the site states of the reaction sites 404. More information regarding the structure and operation of these and other possible components of the HCMC system 400 are provided below.

The digital computer 408 may be configured for controlling, monitoring, executing, and/or coordinating the functions and operations of various components of the HCMC system 400, such as, for example, the reaction site array 402, the site-readout device 410, and the site-manipulation device 412. In particular, the digital computer 408 may be configured for controlling and executing, at least partly, various steps of the HCMC method 300 of FIG. 7 for the purpose of solving the computational problem, as described in greater detail below. The digital computer 408 may be implemented in hardware, software, firmware, or any combination thereof, and be connected to various components of the HCMC system 400 via wired and/or wireless communication links to send and/or receive various types of electrical and digital signals, such as timing and control signals, measurement signals, and data signals. The digital computer 408 may be controlled by direct user input or by programmed instructions, and may include an operating system for controlling and managing various functions of the HCMC system 400. As the case may be, the digital computer 408 may be fully or partly integrated with or physically separate from the other hardware components of the HCMC system 400. In FIG. 1, the digital computer 408 generally includes a processor 414 and a memory 416. The memory 416, which may also be referred to as a “computer readable storage medium”, has stored thereon computer readable instructions that, when executed by the processor 414, cause the processor 414 to perform various steps of the HCMC method disclosed herein, as described in greater detail below.

The processor 414 may implement operating systems, and may be able to execute computer programs, also known as commands, instructions, functions, processes, software codes, executables, applications, and the like. It should be noted that although the processor 414 in FIG. 8 is depicted as a single entity for illustrative purposes, the term “processor” should not be construed as being limited to a single processor, and accordingly, any known processor architecture may be used. In some implementations, the processor 414 may include a plurality of processing units. Such processing units may be physically located within the same device, or the processor 414 may represent the processing functionalities of a plurality of devices operating in coordination. For example, the processor 414 may include or be part of one or more of a computer; a microprocessor; a microcontroller; a coprocessor; a central processing unit (CPU); an image signal processor (ISP); a digital signal processor (DSP) running on a system on a chip (SoC); a single-board computer (SBC); a dedicated graphics processing unit (GPU); a special-purpose programmable logic device embodied in hardware device, such as, for example, a field-programmable gate array (FPGA) or an application-specific integrated circuit (ASIC); a digital processor; an analog processor; a digital circuit designed to process information; an analog circuit designed to process information; a state machine; and/or other mechanisms configured to electronically process information and to operate collectively as a processor.

The memory 416, which may also be referred to as a “computer readable storage medium” is capable of storing computer programs and other data to be retrieved by the processor 414. The terms “computer readable storage medium” and “computer readable memory” refer herein to a non-transitory and tangible computer product that can store and communicate executable instructions for the implementation of various steps of the techniques disclosed herein. The computer readable memory may be any computer data storage device or assembly of such devices, including a random-access memory (RAM); a dynamic RAM; a read-only memory (ROM); a magnetic storage device, such as a hard disk drive, a solid state drive, a floppy disk, and a magnetic tape; an optical storage device, such as a compact disc (CD or CDROM), a digital video disc (DVD), and a Blu-Ray™ disc; a flash drive memory; and/or any other non-transitory memory technologies. A plurality of such storage devices may be provided, as can be appreciated by those skilled in the art. The computer readable memory may be associated with, coupled to, or included in a processor configured to execute instructions contained in a computer program stored in the computer readable memory and relating to various functions associated with the processor.

In some implementations, the HCMC system 400 may include a user interface and a display interface (not shown) operatively coupled to the digital computer 408 and from which aspects or features of the present techniques may be accessed and controlled. The user interface and the display interface may allow the input of commands and queries to the HCMC system 400, as well as present the outcomes of the commands and queries.

Returning to FIG. 7, the HCMC method 300 also includes a step 306 of performing, until an end condition has been met, one or more iterative cycles toward a solution to the computational problem. It is appreciated that the discussion above regarding the terms “solution” and “end condition” generally applies to the iterative cycles performed in the HCMC approach.

In FIG. 7, each iterative cycle includes a step 308 of reading out the site states of the reaction sites with the array in an initial array configuration. In the HCMC system of FIG. 8, the site-readout device 410 may be configured, under control by the digital computer 408, to read out the site states of the reaction sites 404 in the initial array configuration, and the digital computer 408 may be configured to receive, from the site-readout device 410, the read-out site states in the initial array configuration.

It is appreciated that in some embodiments, the HCMC method 300 may include, prior to performing the first iterative cycle, a step of initializing the reaction site array in the initial array configuration. In such a case, the initial array configuration for the first iterative cycle may be random, predetermined, or a combination thereof. In the HCMC system of FIG. 8, the initialization of the reaction site array 402 may be performed with the site-manipulation device 412. In other embodiments, however, the HCMC method 300 may not include a specific step of initializing the reaction site array prior to performing the first iterative cycle. In such a case, the initial array configuration for the first iterative cycle may correspond to whichever configuration the array of reaction sites is into at the beginning of the first iterative cycle. As noted above for the PMC approach, for the iterative cycle, the initial array configuration may or may not be characterized by having each reaction site in one of its possible site states.

Depending on the physicochemical property used, the readout step 308 may be carried out using a variety of readout techniques including, but not limited to, an optical readout scheme, an electrical readout scheme, an electrochemical readout scheme, and any combination thereof. As in the PMC approach, the readout step 308 may include a step of processing or analyzing the physicochemical measurements in order to map them to the possible site states. In the HCMC system 400 of FIG. 8, the processing steps involved in determining the read-out site states from the physicochemical measurement data may be carried out by the site-readout device 410, the digital computer 408, or both. The discussion above regarding various aspects and features relating to the readout of site states in the PMC approach generally applies to the HCMC approach.

Returning to FIG. 7, each iterative cycle also includes a step 310 of determining a candidate solution based on the initial array configuration by inputting the read-out site states for the problem variables in the problem Hamiltonian, followed by a step 312 of determining whether the end condition has been met. In general, the set of read-out site states directly provides the candidate solution to the computational problem associated with a given iterative cycle. As for the PMC approach, the step 312 of determining whether the end condition has been met may include, but is not limited to, any, some, or all of the following steps: assessing whether a specified number of iterative cycles has been completed; assessing whether a specified allowed computation time has been reached; assessing whether the candidate solution meets a specified solution criterion. In particular, the examples of solution criterion mentioned above with respect to the PMC approach generally may generally be applied to the HCMC approach.

It is appreciated that in an iterative process such as the one used in the HCMC method 300 of FIG. 7, multiple candidate solutions may be determined sequentially over multiple iterative cycles. For example, a first candidate solution may be determined in a first iterative cycle, a second candidate solution may be determined in a second iterative cycle, and so on, until the end condition is met. In many iterative processes, the quality of the candidate solutions may be expected, on average, to improve over successive iterative cycles, notably over the first few iterative cycles. The rate at which the quality of the candidate solutions improves over successive iterative cycles may depend on various factors, but eventually one candidate solution may converge to an acceptable solution. The end condition will typically not be met in the first iterative cycle or the first few iterative cycles, although this is not a priori excluded. For example, in some embodiments, between ten and about hundreds of iterative cycles may be performed before meeting the end condition, although other embodiments may operate outside this range.

If, at a given iterative cycle, the end condition has been met, the method 300 of FIG. 7 includes a step of terminating the one or more iterative cycles.

If, at a given iterative cycle, the end condition has not been met, the HCMC method 300 of FIG. 7 includes a step 314 of determining, based on the problem parameters, state perturbations to be applied to a number of the reaction sites 404 to promote state changes therein. In order to allow state changes in the reaction sites 404, it is appreciated that in addition to its the ability to be mapped onto a set of discrete and resolvable site states, the physicochemical property of a given reaction site 404 can also be reversibility changed between the different site states in response to the applied state perturbations.

As noted above, in contrast to the PMC approach, the reaction sites 404 in the HCMC approach generally do not have intra-site couplings and inter-site couplings directly or physically enforced thereon and encoding the problem parameters of the problem Hamiltonian representing the computational problem to be solved. Rather, the HCMC approach accounts for the constraints imposed by the problem parameters by performing calculations on the digital computer 408. The set of read-out site states obtained at the current iterative cycle may be fed to the digital computer 408. The digital computer 408 may be configured to perform calculations on the problem Hamiltonian to determine, based on the read-out site states and the problem parameters, state perturbations to be applied to a number of the reaction sites 404 to promote site changes therein. The state perturbations may represent stepwise instructions or directives, for example, in the form of energy gradients, to be imparted to the reaction sites 404 to favor or induce changes in their site states in an attempt to gradually bring the reaction site array 402 to a configuration that provides an acceptable solution to the computational problem. In some embodiments, a state perturbation may be characterized by a magnitude and a direction. In general, state perturbations are to be applied only to a subset of the reaction sites, the subset generally being different for different iterative cycles depending on the current configuration of the reaction site array 402. In some embodiments, the number of reaction sites to which state perturbations are applied may generally gradually decrease from one iterative cycle to the next. In particular, a finding that there are no more sites to perturb may be indicative of the reaction site array 402 having reached a local, and possibly global, minimum. Once this is the case, the one or more iterative cycles may be terminated.

In some embodiments, the step 314 of determining the state perturbations can include performing a computational optimization operation. It is appreciated that a variety of computational optimization techniques algorithms may be used to compute the state perturbations from the read-out site states and the problem Hamiltonian, including, but not limited to: algorithms with a fixed number of steps; iterative methods that converge to a solution; heuristics that give approximate solutions (not necessarily converging to a global optimum). Non-limiting examples include, to name a few, simulated annealing; stochastic gradient descent; the simplex algorithm; basin-hopping; local search; evolutionary algorithms, including genetic algorithms; and the Hopfield network. It is appreciated that the theory and applications of computational optimization techniques are generally known in the art, and need not be described in detail herein other than to facilitate an understanding of the present techniques. In particular, it is appreciated that those skilled in the art can, based on the teachings of the present disclosure, select or devise an appropriate computational optimization technique to compute state perturbations in accordance with the present techniques. It is noted that an in silico model of an HCMC system that uses a stochastic gradient descent approach is described below in the section entitled “In silico model of an HCMC system”.

As noted above, in the case of the PMC approach, each reaction site may be coupled only to those reaction sites with which it can physically interact, so that some embodiments in the PMC approach may be limited to two-body nearest-neighbor interactions. These limitations related to sparse connectivity and reduced locality may restrict the types of problem Hamiltonians that can be encoded into the reaction site array, and thus the types of computational problems that can be solved, although techniques exist to overcome or go around these limitations, as described above. In contrast, because the HCMC approach enforces problem parameters digitally rather than physically, by using a digital computer 408 instead of enforcing intra-site and inter-site couplings on the reaction sites 404, the HCMC approach may provide full and unlimited connectivity between the reaction sites 404. In particular, the computation by the digital computer 408 of the state perturbations to be applied to the reaction sites need not consider the actual spatial arrangement of the reaction sites 404 in the array 402, so that the problem parameters of the problem Hamiltonian can include k-local and/or non-nearest-neighbor interactions.

Referring to both FIGS. 7 and 8, once the state perturbations to be applied to the number of the reaction sites 404 have been determined by the digital computer 408, each iterative cycle the HCMC method 300 includes a step 316 of controlling, by the digital computer 408, the site-manipulation device 412 to apply the state perturbations to the number of the reaction sites to promote state changes therein. It is appreciated that various types of site-manipulation devices 412 may be used, for example, based on optical, electrical, and/or electrochemical actuation and control schemes. In one embodiment, the site-manipulation device 412 may include an electrode array with associated electronic and control circuitry (see, e.g., FIG. 5 and the section below entitled “Physical implementation of the HCMC approach”). In another embodiment, the site-manipulation device 412 may include a spatial light modulator (SLM) configured to illuminate the reaction site array 402 with controllable spatio-temporal illumination patterns that can allow the state perturbations to be applied on a per-site basis. Other non-limiting examples include, to name a few: using a laser or SLM to trigger photochemical reactions (e.g., photoredox reactions), applying an electrical current to a droplet to a drive a redox reaction; applying an electrical stimulation to change the electrical conductance or resistance; and applying a mechanical stimulation (e.g., by shaking, stirring, or applying pressure to increase diffusion; or using a robotic arm with a syringe to inject a new or additional substance to a droplet). It is appreciated that various techniques are known in the art to initialize, activate, change, control, or otherwise act on droplets, which may be used in the embodiments herein.

Once the state perturbations have been applied to the relevant reaction sites 404 by the site-manipulation device 412, each iterative cycle further includes a step 318 of allowing the array 402 to evolve, from the initial array configuration and responsive to the applied state perturbations, to a final array configuration in which each reaction site 404 is in one of its at least two possible site states. It is appreciated that in the HCMC approach, the reaction sites 404 are generally not connected to one another, such that each reaction site 404 having a state perturbation applied thereto will evolve under the state perturbation independently of the evolution of the other such reaction sites 404. However, in other embodiments, there may exist some degree of coupling between the reaction sites 404 (e.g., by enforcing intra-site and/or inter-site couplings as in the PMC approach). In such embodiments, the final array configuration will depend not only on the state perturbations applied to the reaction sites 404, but also on the couplings between the reaction sites 404. It is also appreciated that the HCMC system 400 does not know beforehand the final array configuration it will end up in as a result of the applied state perturbations. Rather, the digital computer 408 merely imposes, via the site-manipulation devices, the state perturbations to a subset of reaction sites 404 to promote or favor changes in the site states of these reaction sites 404. As noted above, depending on the nature and complexity of the computational problem, the problem Hamiltonian may have a single ground state corresponding to a single optimal final array configuration, a plurality of degenerate ground states corresponding to a plurality of optimal final array configurations, or one or more local minima that the PMC system identifies as “sufficiently good” solutions. The amount of time taken by the HCMC system 400 to evolve, at each iterative cycle, from an initial to a final array configuration may vary depending on the application.

Once the final array configuration has been reached, each iterative cycle of the HCMC method 300 includes a step 320 of setting the final array configuration as the initial array configuration for the next iterative cycle. This means that the final array configuration obtained at the i^{th} iterative cycle will be read out (step 308) as the initial array configuration of the (i+1)^{th} iterative cycle to determine a new candidate solution (step 310) and to assess whether the end condition has been met (step 312). If it is assessed that the one or more iterative cycles may be terminated without performing steps 314, 316, 318, and 320 for the (i+1)^{th} iterative cycle.

Once the one or more iterative cycles have been terminated, the HCMC method 300 of FIG. 7 includes a step 322 of determining the solution to the computational problem from at least one of the one or more candidate solutions. As noted above for the PMC approach, it is appreciated that if only one candidate solution is determined, corresponding to only one iterative cycle having been performed at step 306, then the solution to the computational problem is determined from the single candidate solution. However, if multiple candidate solutions have been determined, corresponding to multiple iterative cycles having been performed at step 306, then the solution to the computational problem is determined from at least one of the multiple candidate solutions. In some embodiments, the solution to the computational problem may correspond to a particular one of the multiple candidate solutions, for example, the candidate solution obtained at the last iterative cycle or at one of the last few iterative cycles. In one example, the particular candidate solution may correspond to a ground state or a suitable approximation of a ground state of the problem Hamiltonian encoding the computational problem. In another example, the particular candidate solution may correspond to a best feasible solution to the computational problem. In yet another example, the particular candidate solution may provide a higher or best match among the multiple candidate solutions or a subset of the multiple candidate solutions. In other embodiments, the solution to the computational problem may correspond to a subset of the multiple candidate solutions, for example, if the problem Hamiltonian encoding the computational problem has a subset of degenerate ground states.

It is appreciated that performing one instance of the one or more iteration cycles (i.e., one instance of step 306 in the embodiment of FIG. 7) may define one computational run in the HCMC method 300. In such a case, the solution obtained from the one or more candidate solutions obtained during the run may be referred to as a single-run solution. In some embodiments, the HCMC method 300 may include performing multiple instances of step 306 to obtain multiple single-run solutions, and determining, at step 322, the solution to the computational problem from the multiple single-run solutions.

In accordance with another aspect, there is provided a non-transitory computer readable storage medium having stored thereon computer readable instructions that, when executed by a processor, cause the processor to perform a method for solving a computational problem using an array of reaction sites. The computational problem is represented as a problem Hamiltonian expressed in terms of a set of problem variables and a set of problem parameters. The array of reaction sites has a physicochemical property that maps to at least two possible site states, and each reaction site represents one of the problem variables. The method may include a step of performing, by the processor, one or more iterative cycles toward a solution to the computational problem. Each iterative step may include steps of receiving the site states of the reaction sites read out in an initial array configuration, determining a candidate solution to the computational problem from the initial array configuration by inputting the read-out site states for the problem variables in the problem Hamiltonian, and determining whether an end condition has been met. If the end condition has been met, the method may include terminating the one or more iterative cycles. If the end condition has not been met, each iterative cycle may include steps of determining, based on the problem parameters, state perturbations to be applied to a number of the reaction sites to promote state changes therein, controlling a site-manipulation device to apply the state perturbations, allowing the array to evolve, from the initial array configuration and responsive to the applied state perturbations, to a final array configuration in which each reaction site is in one of the at least two possible site states, the final array configuration; and setting the final array configuration as the initial array configuration for the next iterative cycle. Once the one or more iterative cycles have been terminated, the method may include a step of determining, by the processor, the solution to the computational problem from at least one of the one or more candidate solutions. It is appreciated that the method performed by the processor may include various steps, aspects, and features of the HCMC method described herein.

In accordance with another aspect, there is provided a digital computer including a processor; and a non-transitory computer readable storage medium operatively coupled to the processor and having stored thereon computer readable instructions that, when executed by a processor, cause the processor to perform various steps of the HCMC method described herein. FIG. 8 depicts an example of a digital computer 408 that includes a processor 414 and a non-transitory computer readable storage medium 416 (also referred to above as a memory) operably connected to the processor 414.

### Examples & Applications

The following description reports work conducted to study and investigate various aspects of the present techniques. It is appreciated that the problem solving techniques described herein may have a number of optional features, variations, and applications. In particular, the following description is provided to further illustrate some aspects of the disclosed techniques, but should not be construed as in any way limiting their scope. Some of these aspects were reporting in the following article, the entire contents of which are incorporated herein by reference: S. Y. Guo, P. Friederich, Y. Cao, T. Wu, C. Forman, D. Mendoza, M. Degroote, A. Cavell, V. Krasecki, R. Hickman, A. Sharma, L. Cronin, N. Gianneschi, R. Goldsmith, and A. Aspuru-Guzik, “A Molecular Computing Approach to Solving Optimization Problems via Programmable Microdroplet Arrays”, ChemRxiv: 10,250,897, (2019).

### Boolean Satisfiability: Conjunctive Normal Formula to Ising Hamiltonian

An example of how to map a computational problem to an Ising Hamiltonian is presented. A k-SAT Boolean formula of N variables consists of M clauses of k literals each. An example of a 3-variable 2-SAT in conjunctive normal form (CNF) is:

(x or y) and (y and not z). (5)

To convert this expression into an Ising Hamiltonian, each Boolean variable may be represented as a spin, sᵢ∈{−1, +1}, i=1, . . . , N. First, a clause matrix may be defined as:

$$\{ \begin{matrix}{w}_{ji} = 1 , if clause j includes {s}_{i} \\ {w}_{ji} = - 1 , if clause j i ncludes not {s}_{i}\end{matrix} . \qquad ( 6 )$$

Each clause indicator may be written as:

$${v}_{j} = \frac{1}{{2}^{k}} {\prod }_{i = 1}^{N} ( 1 - {w}_{ji} {s}_{i} ) . \qquad ( 7 )$$

The overall Hamiltonian becomes:

$${H}_{k - SAT} = {\sum }_{j = 1}^{M} {v}_{j} = \frac{1}{{2}^{k}} {\sum }_{j = 1}^{M} {\prod }_{i = 1}^{N} ( 1 - {w}_{ji} {s}_{i} ) . \qquad ( 8 )$$

When the CNF is satisfied, H_{k-SAT}=0. For H_{k-SAT}>0, the value of H_{k-SAT} gives the number of violated causes. To use binary variables xᵢ∈{0, 1} instead of spin variables, the following transformation may be used:

$${s}_{i} = ( 2 {x}_{i} - 1 ) or {x}_{i} = \frac{1}{2} ( {s}_{i} + 1 ) . \qquad ( 9 )$$

### Four-Droplet System

To illustrate how the molecular computing approach disclosed herein may work, the following satisfiability problem of five clauses, each with two variables (2-SAT), is considered. Although the 2-SAT problem is in P and not NP, it provides a convenient illustration how a molecular computing system may operate. In CNF representation:

(s₁ or s₂) and (s₂ or not s₃) and (s₃ or s₄) and (not s₄ or s₁) and (not s₂ or not s₄) or (s₁∨s₂)∧(s₂∨!s₃)∧(s₃∨s₄)∧(!s₄∨s₁)∧(!s₂∨!s₄). (10)

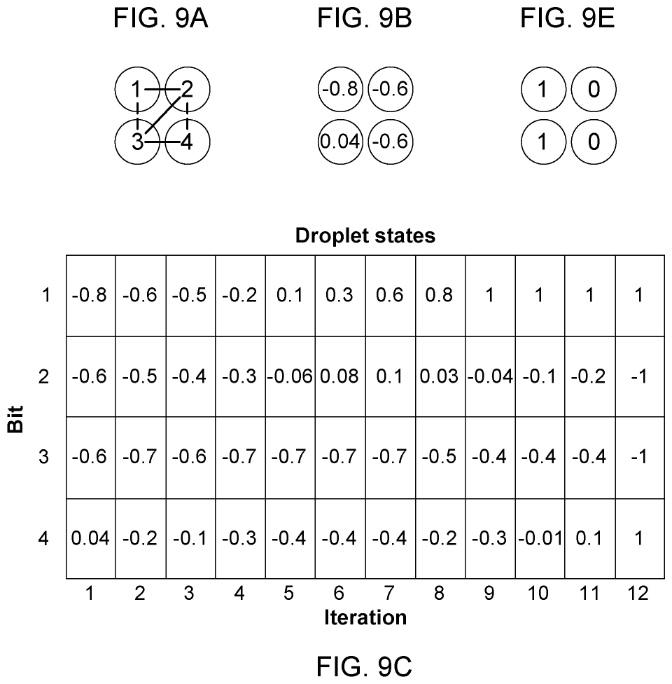

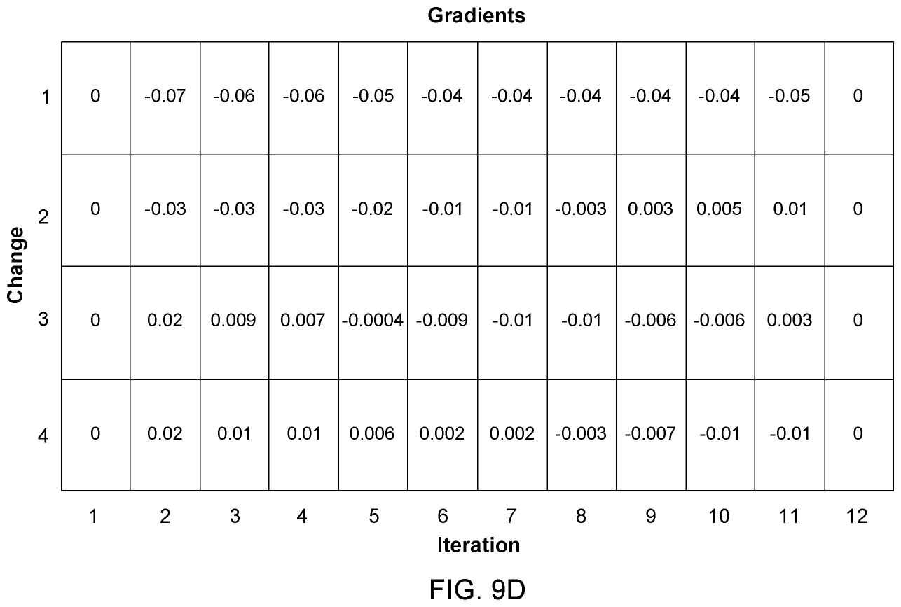

FIGS. 9A to 9E illustrate how this calculation may be implemented in a four-droplet system, using a simulation of an HCMC system such as disclosed herein. In FIG. 9A, the clauses are indicated by lines connecting the droplets (solid line: “positive” coupling; dashed line: “negative” coupling). The problem may be mapped to an Ising Hamiltonian according to the procedure described in the section entitled “Boolean satisfiability: conjunctive normal formula to Ising Hamiltonian”.

$${H}_{P} = {h}^{( 0 )} + {\sum }_{i} {h}_{i}^{( 1 )} {s}_{i} + {\sum }_{( i , j )} {h}_{i j}^{( 2 )} {s}_{i} {s}_{j} , \qquad ( 11 )$$

$${h}^{( 0 )} = \frac{5}{4} , \qquad ( 12 )$$

$${h}^{( 1 )} = \frac{1}{4} \begin{matrix}[ - 2 & - 1 & 0 & 1 ] ,\end{matrix} \qquad ( 13 )$$

$${h}^{( 2 )} = \frac{1}{8} [ \begin{matrix}0 & 1 & 0 & - 1 \\ 1 & 0 & - 1 & 1 \\ 0 & - 1 & 0 & 1 \\ - 1 & 1 & 1 & 0\end{matrix} ] . \qquad ( 14 )$$

These arrays are entered into the control algorithm of a computer software. To run a calculation, each droplet state is first randomly initialized (FIG. 9B). The control algorithm takes in the droplet states (FIG. 9C), then computes the gradients on each droplet based on the problem Hamiltonian above (FIG. 9D). These gradients are delivered to the droplets via changes in electric potential, for example. Over a number of iterations, the system reaches a minimum energy state and stops when the gradients reach zero. The final state is rounded to the nearest integer value (i.e., −1 or +1) and the result is read out, after a change of basis from +1 to 0, 1 (FIG. 9E). In this example, a solution s₁=1, s₂=0, s₃=0, and s₄=1 satisfies all of the clauses given in Eq. (9).

### Mapping Bits to an HCMC and a PMC for Nine Variables

An example is given of how a Boolean satisfiability problem can be mapped to an HCMC and a PMC implemented on two different lattices. For the purposes of more easily illustrating the relationships between the variables, a 2-SAT problem (9 variables, 17 clauses) is used. The CNF representation is:

(1∨4)∧(!2∨5)∧(!3∨!9)∧(!2∨4)∧(!3∨5)∧(6∨9)∧(!3∨7)∧(!4∨8)∧(2∨9)∧(7∨!8)∧(!2∨!6)∧(1∨6)∧(!4∨5)∧(2∨5)∧,(!4∨9)∧(3∨4)∧(!2∨3) (15)

which can be represented as an Ising Hamiltonian with the bias, one-body, and two-body term coefficients as follows:

$${H}_{P} = {h}^{( 0 )} + {\sum }_{i} {h}_{i}^{( 1 )} {s}_{i} + {\sum }_{( i , j )} {h}_{i j}^{( 2 )} {s}_{i} {s}_{j} , \qquad ( 16 )$$

$${h}^{( 0 )} = \frac{17}{4} , \qquad ( 17 )$$

$${h}^{( 1 )} = \frac{1}{4} [ \begin{matrix}- 2 & 2 & 1 & 0 & - 4 & 1 & - 2 & 0 & 0\end{matrix} ] , \qquad ( 18 )$$

$${h}^{( 2 )} = \frac{1}{8} [ \begin{matrix}0 & 0 & 0 & 1 & 0 & 1 & 0 & 0 & 0 \\ 0 & 0 & - 1 & - 1 & 0 & 1 & 0 & 0 & 1 \\ 0 & - 1 & 0 & 1 & - 1 & 0 & - 1 & 0 & 1 \\ 1 & - 1 & 1 & 0 & - 1 & 0 & 0 & - 1 & - 1 \\ 0 & 0 & - 1 & - 1 & 0 & 0 & 0 & 0 & 0 \\ 1 & 1 & 0 & 0 & 0 & 0 & 0 & 0 & - 1 \\ 0 & 0 & - 1 & 0 & 0 & 0 & 0 & - 1 & 0 \\ 0 & 0 & 0 & - 1 & 0 & 0 & - 1 & 0 & 0 \\ 0 & 1 & 1 & - 1 & 0 & - 1 & 0 & 0 & 0\end{matrix} ] . \qquad ( 19 )$$

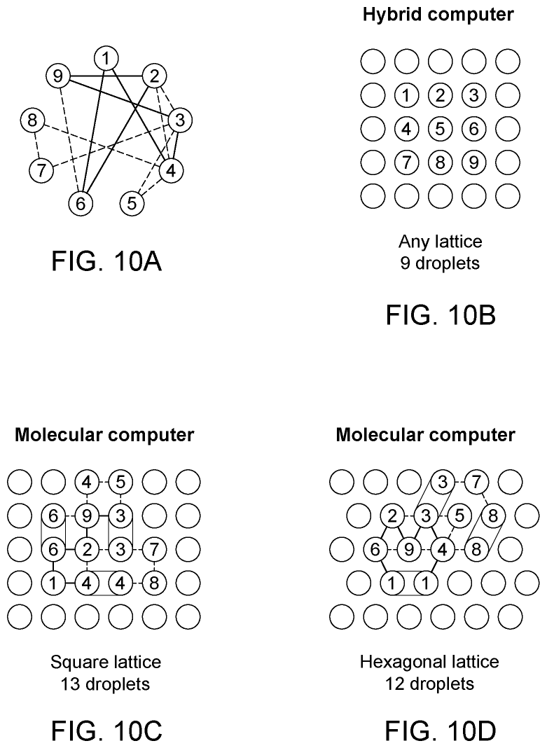

In FIG. 10A, the problem is represented as a graph (solid line: “positive” coupling; dashed line: “negative” coupling). FIGS. 10B to 10D illustrate how the problem may be physically placed on a droplet array for an HCMC (FIG. 10B), a PMC with a square lattice (FIG. 10C), and a PMC with a hexagonal lattice (FIG. 10D). Since the HCMC is fully connected and relies on a classical computer to enforce inter-droplet couplings, the droplets may be placed anywhere, in any order. For the PMC, depending on the lattice (e.g., square or hexagonal), each droplet can be coupled to four or six nearest-neighbor droplets. As noted above, to overcome the spatial and connectivity constraints, minor embedding techniques and ancillary variables may be used. For example, the square lattice in FIG. 10C uses an extra droplet for each of variables 3 and 6 and two extra droplets for variable 4, while the hexagonal lattice in FIG. 10D uses an extra droplet for each of variables 1, 3, and 8. Although this approach invariably increases the size of the system, it can ensure that all droplets can be pairwise coupled.

### In Silico Model of an HCMC System

The HCMC approach disclosed herein may be implemented using an array of microdroplets containing various chemical compounds. Droplet manipulation may be achieved with electrostatic fields and electrochemical transformations induced by electrodes or by optical stimulation induced by light sources. Changes in droplet states may be measured and detected optically, which may allow for a convenient readout of the states. For simplicity, an abstract nomenclature of states 0 and 1 is used in for the present example. Droplets are assumed to fluctuate within this range of states as a result of external stimuli controlled by a classical computer. The value associated with each droplet may be thought of as the droplet's progress along an arbitrary reaction coordinate. To explore the capabilities and potential application areas of the HCMC approach, an in silico model of an HCMC system was implemented. The in silico model included two parts: a set of readable and writable states that represents an array of droplets in the HCMC system; and a computer algorithm configured to receive a problem Hamiltonian encoding a computational problem to be solved and to use the problem Hamiltonian to calculate and apply stepwise changes to the set of states toward a solution to the computational problem.

The in silico model of an HCMC system was applied to the following three example applications: a Boolean satisfiability problem, a lattice protein folding problem, and a traveling salesperson problem. These examples are described below. Solving these computational problems in the HCMC approach disclosed herein involved the following steps: mapping the computational problem to an Ising Hamiltonian; initializing the droplets in random states; encoding the bias parameters and the interaction parameters of the Ising Hamiltonian in the intra-droplet and inter-droplet couplings; using an optimization method to find a local optimum, from which a solution to the computation problem may be read out and checked on validity. Repeating these steps for a number of cycles may allow for the determination of whether the computational problem has a solution that fulfills all conditions (e.g., in the case of a Boolean satisfiability problem) or that identifies low-energy conformations of a protein (e.g., in the case of a lattice protein folding problem).

In the studied in silico HCMC model, a simulated annealing process was used as an optimization method. The simulated annealing process was modeled using a stochastic gradient descent approach. As described below, computational problems, including Boolean satisfiability, lattice protein folding, and travelling salesperson problems, may be expressed as Ising Hamiltonians represented in the form of tensors h⁽⁰⁾, h⁽¹⁾, . . . , h⁽ⁿ⁾. In this representation, the term h⁽⁰⁾ is an offset to the global energy that does not influence the states of the system, the term h⁽¹⁾ is a vector of bias terms representing intra-droplet couplings, the term h⁽²⁾ is a matrix of two-body interaction terms representing two-droplet couplings, the term h⁽³⁾ is a rank-3 tensor of three-body interaction terms representing three-droplet couplings, and so on. For example, the 3-SAT problem and the lattice protein folding problem described below may be converted to an Ising Hamiltonian with up to three-body and four-body interaction terms, respectively. As discussed above, the HCMC approach disclosed herein can handle interactions of any order, with n droplets for n-body terms, though it is possible to convert higher-order interactions, such as three-body and four-body interactions, into two-body interactions by introducing ancillary states in the system.

To simulate the annealing process of a droplet array, an iterative procedure was used. Each iteration involved reading out the states of the system; calculating the gradients acting on the states in terms of bias potentials (i.e., h⁽¹⁾) and many-body interactions (i.e., h⁽²⁾ to h⁽ⁿ⁾); applying changes to a number of the states with a finite step size. The procedure was carried out in two modes: ensemble annealing mode and trajectory mode. The ensemble annealing mode involved running many short annealing simulations from random initial states, and analyzing the statistics of the final states in search for the global optimum. The trajectory mode involved running one annealing simulation over a large number of steps but adding a certain level of noise on the gradients for the purpose of attempting to overcome energy barriers between local optima. For example, in some applications, the HCMC approach disclosed herein could potentially benefit from accelerated sampling methods (e.g., developed for droplet dynamics simulations, such as, to name a few, basin-hopping, metadynamics, and umbrella sampling) to overcome kinetic trapping.

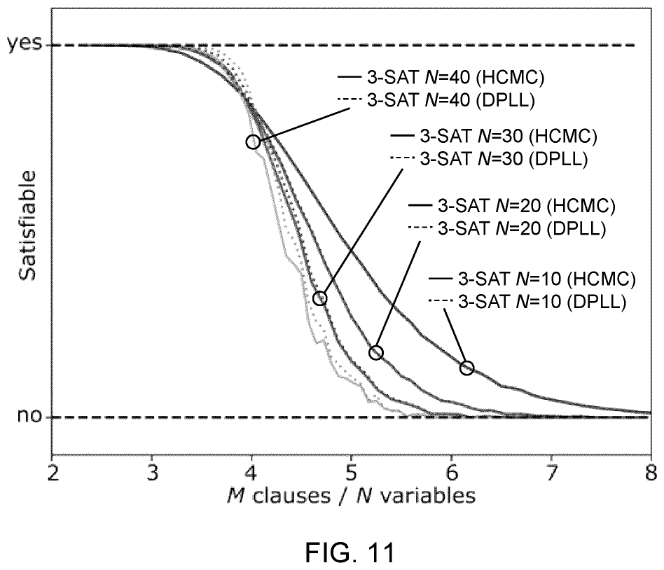

Example 1: Boolean satisfiability. The Boolean satisfiability problem is a problem of determining whether there exists a set of Boolean variables that satisfies a given Boolean formula. The Boolean satisfiability problem is NP-complete. Solving Boolean satisfiability problems such as 3-SAT problems using the HCMC approach disclosed herein may involve converting the problem from its CNF representation to an Ising model (see above). In the case of 3-SAT problems, the Ising model will contain up to three-body coupling terms. The satisfiability of random 3-SAT problems with N variables and M unique clauses of k=3 clauses was tested. The results are shown in FIG. 11, which depicts a graph of the probability of satisfiability as a function of clause density (ratio M/N) for a random 3-SAT problem obtained using a computational model of the HCMC approach. The results are compared to solutions obtained using the Davis-Putman-Logemann-Loveland (DPLL) algorithm (dotted curves). Each point in the graph is the average of up to 10,000 3-SAT problems. The results show a transition from a high probability of a problem being satisfiable when the clause density is small to a low probability when the clause density is large. The results obtained using the HCMC approach are in good agreement with the results obtained using the (exact) DPLL algorithm.

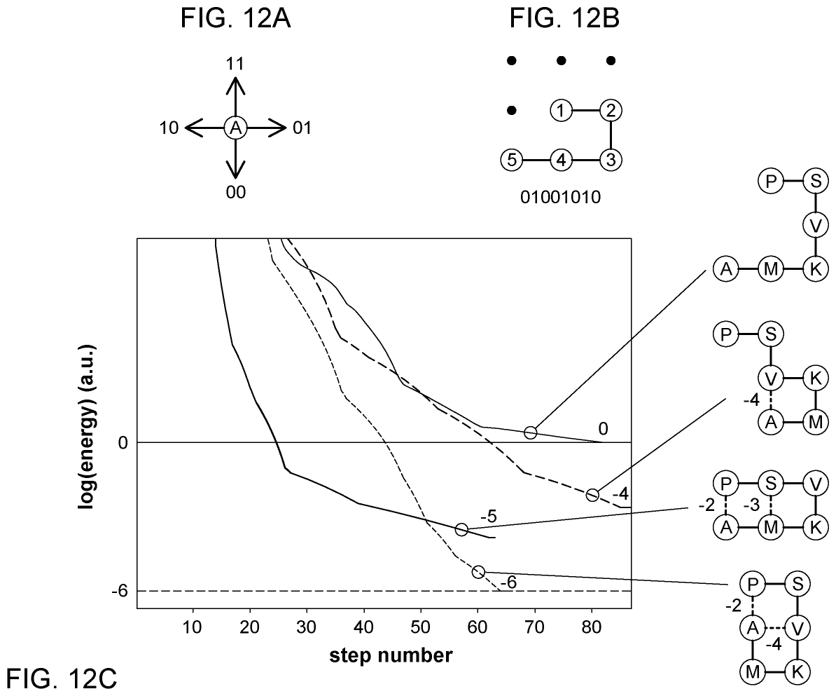

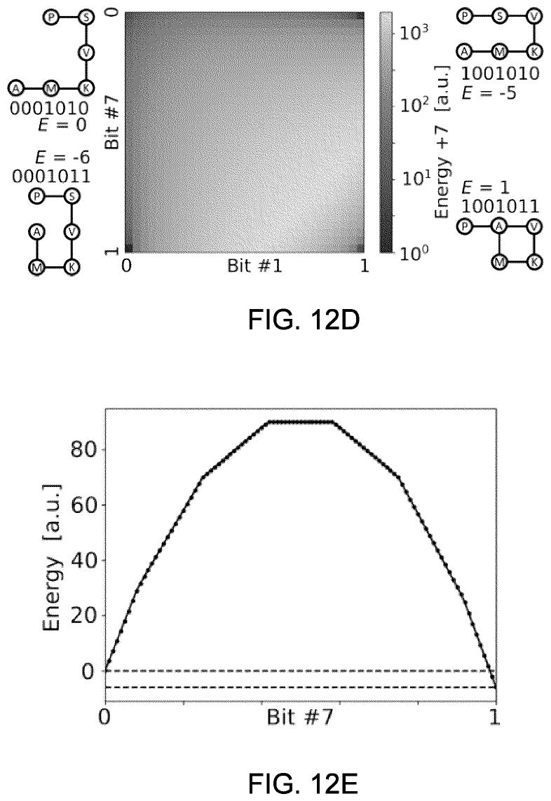

Example 2: Lattice protein folding. A computational model of the HCMC was tested on the two-dimensional lattice protein model. The proteins in this model consist of a sequence of amino acids that can fold on a two-dimensional square lattice. The protein conformation is represented as a sequence of turns, where each turn is encoded by two bits (00: downward; 01: rightward; 10: leftward; 11: upward). FIGS. 12A and 12B depict the two-bit representation of the turns (FIG. 12A) and an example of protein folding (FIG. 12B).

The energy of each conformation includes two terms: clashes of two amino acids are penalized with positive terms of size λ, whereas attracting interactions between specific amino acids (i and j, |i−j|>1) are rewarded with negative energy terms εᵢⱼ in case amino acids i and j are direct, nondiagonal neighbors (native contact). An Ising Hamiltonian with up to four-body interactions may be constructed that encodes both types of interactions. This involves ancillary bits that activate and deactivate depending on the state of the bits that encode the physical conformation of the protein. The overlap parameter λ may be chosen in a way that the energy spectrum of the protein model, and thus of the Ising Hamiltonian, has negative or zero energies for all conformations without clash and positive energies for all other conformers. The global ground state of the Hamiltonian yields the protein conformation in which the highest possible number of native bonds are formed. Non-clashing conformers with fewer or less strong native bonds are local optima with energies smaller than zero, whereas non-clashing conformers without any native bond formed have zero energy, corresponding to unfolded conformations.

As described above, the HCMC approach disclosed herein may be used to solve Ising models with four-body interactions. In the present example, the four-body Ising model was converted to a two-body Ising model, which involved the introduction of additional bits, also referred to as reduction bits. The results of annealing simulations of a lattice protein model with six amino acids are shown in FIG. 12C. The problem Hamiltonian that was solved contained 28 bits coupled with two-body interactions, corresponding to 19 bits coupled with up to four-body interactions. Selected trajectories that ended at low-energy conformations show the energy as a function of the step number. The folded configuration corresponding to each trajectory is depicted. Formation of native contacts, as well as annealing of the ancillary bits, reduce the total energy of the system. The final states depicted in FIG. 12C are local optima of the Hamiltonian. The barriers between these local optima are considerably higher than the energy differences between them, which makes it difficult, if not impossible, for a (stochastic) gradient descent algorithm to overcome the barriers between the local optima and the global optimum. For this reason, finding the ground state of the Hamiltonian, and thus the lowest-energy conformation of the protein, generally involves running many instances of the HCMC model simulation. In this example, the ground state configuration has an energy of −6.

In order to investigate whether a (stochastic) gradient descent model is able to escape local minima, the energy landscape between local minima using constrained optimizations at states between the optima was studied, as depicted in FIGS. 12D and 12. An interpolation was performed between the ground state conformation with an energy of −6 (lower left in FIG. 12D) and a non-clashing, unfolded conformation with an energy of 0 (upper left in FIG. 12D). FIG. 12E is a graph of the energy of the system on a line between the unfolded state 0001010 and the folded state 0001011. These two states differ only by one bit (i.e., bit #7). FIG. 12E illustrates that the energy barrier between the two states is higher than 90, which is considerably more than the differences between all valid conformations (i.e., E=−6 to E=0; see FIG. 12C).

Example 3: Traveling salesperson problem. The present techniques may be used to solve the traveling salesperson problem, which is an NP-hard problem in computational optimization that involves finding the shortest route that visits a set specified locations (e.g., cities) exactly once, starting and ending at the same location. The traveling salesperson problem is an important combinatorial optimization problem in many applications including, to name a few, planning, scheduling, and logistics. For example, Table II lists the distances between all possible pairs of the following cities: Toronto, ON; Glasgow, UK; Evanston, Ill.; and Madison, Wis.

TABLE II
Distances between pairs of cites for TSP.

| Pair of cities | Distance between pair of cities (km) |
|---|---|
| Toronto ↔ Glasgow | 5,278.7 |
| Toronto ↔ Evanston | 702.2 |
| Toronto ↔ Madison | 815.5 |
| Glasgow ↔ Evanston | 5,910.5 |
| Glasgow ↔ Madison | 5,894.6 |
| Evanston ↔ Madison | 183.5 |

The traveling salesperson problem may be expressed as a quadratic problem Hamiltonian as follows:

$${H}_{P} = A [ {\sum }_{v = 1}^{N} {( 1 - {\sum }_{j = 1}^{N} {x}_{v , j} )}^{2} + {\sum }_{j = 1}^{N} {( 1 - {\sum }_{v = 1}^{N} {x}_{v , j} )}^{2} ] + B {\sum }_{u < v} {W}_{u v} {\sum }_{j = 1}^{N - 1} {x}_{u , j} {x}_{v , j + 1} , \qquad ( 20 )$$

where the first term represents a penalty for visiting a city twice, the second term represents a penalty for visiting two cities in one stop, and the third term accounts for the distances between the cities. In the present example, N=4, corresponding to four cities, and x_{u,j} is equal to 16, where x_{u,j}=1 if city u is visited at stop j and 0 otherwise, with u=1 for Toronto, u=2 for Glasgow, u=3 for Evanston, and u=4 for Madison. The inter-city distance matrix Wᵤᵥ is given by:

$${W}_{u v} = [ \begin{matrix}0 & 5 , 278.7 & 702.2 & 815.5 \\ 5 , 278.7 & 0 & 5 , 910.5 & 5 , 894.6 \\ 702.2 & 5 , 910.5 & 0 & 183.5 \\ 815.5 & 5 , 894.6 & 183.5 & 0\end{matrix} ] . \qquad ( 21 )$$

Solving the problem with an array of 16 droplets leads to the following solution:

$$[ \begin{matrix}{x}_{1 , 1} = 0 & {x}_{1 , 2} = 1 & {x}_{1 , 3} = 0 & {x}_{1 , 4} = 0 \\ {x}_{2 , 1} = 1 & {x}_{2 , 2} = 0 & {x}_{2 , 3} = 0 & {x}_{2 , 4} = 0 \\ {x}_{3 , 1} = 0 & {x}_{3 , 2} = 0 & {x}_{3 , 3} = 1 & {x}_{3 , 4} = 0 \\ {x}_{4 , 1} = 0 & {x}_{4 , 2} = 0 & {x}_{4 , 3} = 0 & {x}_{4 , 4} = 1\end{matrix} ] , \qquad ( 22 )$$

which corresponds to the route Glasgow→Toronto→Evanston→Madison, which has a total distance of 6,161.4 km.

### Physical Implementation of the HCMC Approach

Various aspects of a physical implementation of the HCMC approach were tested in a study. It will be appreciated from the overall description of this study that the HCMC systems and methods described herein may have a number of optional features, variations, and applications. In particular, the following description of experimentation and results is provided to further illustrate some aspects of the disclosed principles, but should not be construed as in any way limiting their scope.

Property mapping to droplet states. The studied implementation of an HCMC system used the redox reaction illustrated below between hydroquinone (HQ; reduced) to benzoquinone (BQ; oxidized) as a chemical reaction that displays binary states that are readily resolvable and measured by optical or electrochemical readout.

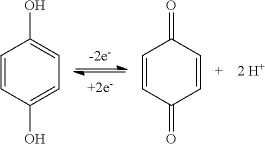 [SMILES: Oc1ccc(O)cc1 ; O=C1C=CC(=O)C=C1]

The oxidation of HQ to BQ at an electrode in solution produces H⁺, thus lowering the pH at the electrode. The corresponding reduction reaction of BQ to HQ consumes H⁺, thus raising the pH at the electrode. This reaction provides access to two states—a high-pH state and a low-pH state—and the ability to switch reversibly between them using electrochemical control. In the studied implementation, the redox reaction was carried in the presence of a small amount of phosphate buffer at pH 7 to provide a neutral starting point. From this neutral starting point, the oxidation or the reduction reaction may be used to set each droplet into a specified initial state at the beginning of computation.

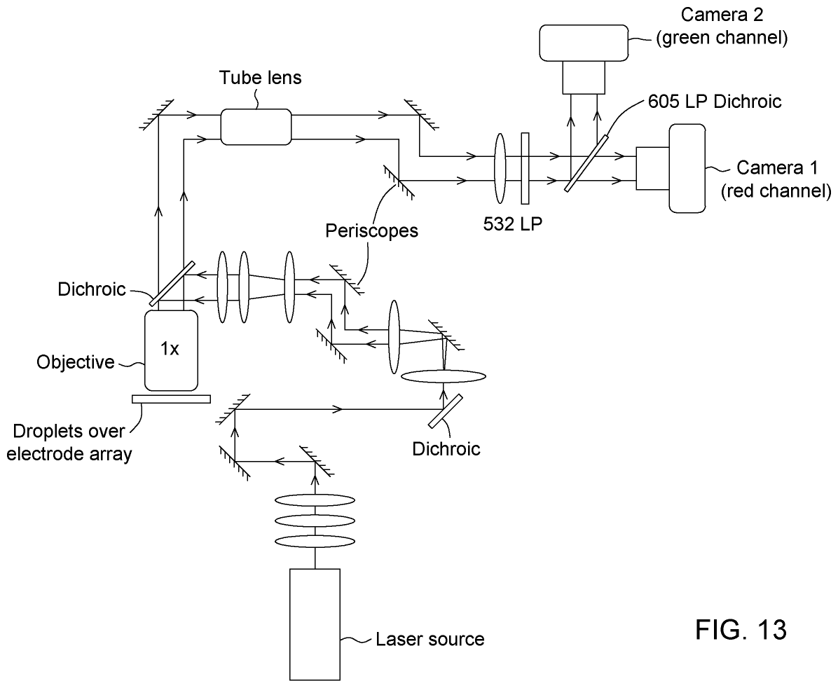

State readout implementations. The disclosed HCMC implementation used a fluorescence microscopy-based optical readout method with pH-sensitive fluorescent probes to read out droplet states. The pH-sensitive fluorophore SNARF™-1 was tested but various other types of fluorescent pH indicators may be used in other applications. By irradiating the droplet array with excitation light (e.g., laser light) and detecting fluorescence from the pH-sensitive probes in response to the excitation light, a fluorescence image of the droplet array can be obtained in which droplet states can be determined from the emitted fluorescence intensity of the different pH-sensitive probes. The fluorescence microscope that was used in the study is depicted schematically in FIG. 13. A ratiometric method was used in which the fluorescence emitted by the SNARF-1 fluorophores was split by a 605-nm long-pass dichroic mirror and measured in two wavelength bands to define two detection channels. SNARF-1 fluorophores have two emission peaks, one above and the other below 605 nm, whose respective intensities vary with pH in opposite fashion. By measuring both detection channels simultaneously, the ratio of the measured intensities can be used to determine the actual pH change at an electrode. The use of a ratiometric approach may improve sensitivity at this approach may be less sensitive to noise, photobleaching, or other issues related to absolute fluorescence measurements.

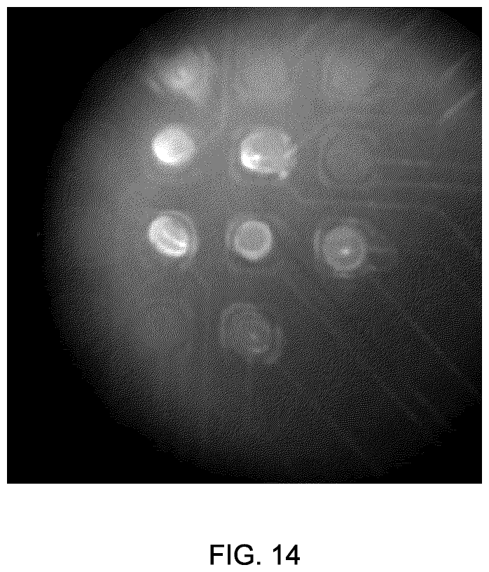

FIG. 14 depicts a fluorescence image of droplets disposed on an array of electrodes. The droplets were composed of water; poloxamer 407, gelling agent used to stabilize the droplets; a HQ-BQ mixture; phosphate buffer for pH stabilization; and the pH-sensitive fluorophore SNARF™-1. The gelling agent was used to slow diffusion enough to allow computations to run in a continuous gel solution (i.e., without separate droplets), as the changes in droplet states at each electrode remain confined to that area over sufficiently long timescales. It is appreciated that while FIG. 14 depicts a 2×2 droplet array, the disclosed readout method is, in principle, scalable to arbitrarily large arrays by adjusting the optical readout setup.

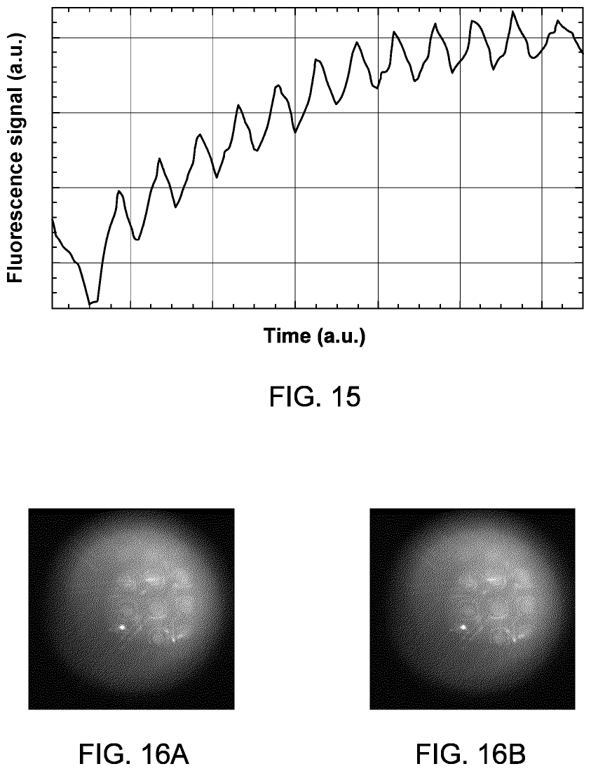

By scanning the voltage at an electrode back and forth as a function of time, it was possible to change the pH of the corresponding droplet in accordance with the alternating oxidation and reduction cycles driven by the applied voltage, and to monitor these pH changes as time variations in the intensity of the fluorescence signal received from the electrode. FIG. 15 is a graph of the fluorescence signal measured at an electrode as the voltage at the electrode was scanned back and forth between 0 V and 5V over many cycles. Furthermore, FIGS. 16A and 16B are fluorescence images of a droplet array having different voltages applied to the working electrodes (FIG. 16A: 0 V applied to all working electrodes; FIG. 16B: −5 V applied to all working electrodes). FIGS. 16A and 16B illustrate that the intensity of the fluorescence emitted by pH-sensitive fluorophores contained in the droplets can be controlled by electrically driving redox-induced pH changes in the droplets.

Electrode control implementations. A multiplexed potentiostat from IO Rodeo was used to provide independent control over seven working electrodes in an array (along with multiple counter electrodes sharing a common control). It is appreciated that a combination of multichannel potentiostats with multiplexing could be used to control significantly larger droplet arrays than those considered in the present study.

Software implementations. A custom code written in Python was used that, when executed by the processor, cause the processor to perform steps of selecting a problem Hamiltonian for solving a computational problem of interest; computing initial states for each droplet; and controlling voltages to be applied to each electrode in order to change the pH at the electrode until the computed initial droplet state is achieved, that is, until fluorescence measurements produced intensity ratios that match the computed initial state. The code is also configured to receive the measured states and feeds them into the next step of the calculation; determine the direction that the pH of each droplet/electrode should go; and apply new voltages as state perturbations to promote these pH changes. The code is configured to repeat these steps—readout of droplet states, determination of state perturbation from read-out droplet states, application of voltages to enforce the state perturbations on the droplet states—in a loop until the system converges to a solution to the problem. In some cases, finding the solution to a given problem, or finding all of the solutions if the problem has multiple solutions, may involve performing multiple runs of the loop.

Operation of the HCMC and results. In one experiment, samples were prepared as solutions of 30% poloxamer 407 in water with 25 mM BQ/HQ, 10 mM phosphate buffer at pH 7, and 25 μM SNARF-1 pH indicator. In order to remain fluid, the solution were kept at a temperature below 20° C., which is less than the sol-gel transition temperature of poloxamer 407. The droplets were disposed in an array using a droplet robot. Continuous gels were pipetted onto the array and flattened with a top coverslip to make imaging easier.

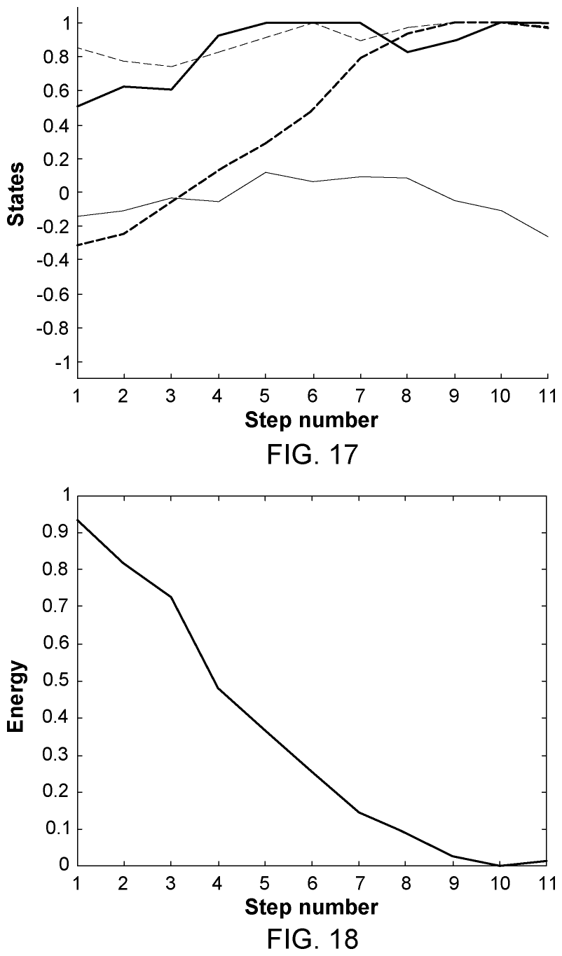

During computations, fluorescence images of the array were acquired at an image acquisition rate of about 1 kHz. Each fluorescence image was analyzed to determine the fluorescence ratio at each droplet, the fluorescence ratio being indicative of the pH of the droplet. The fluorescence ratios were normalized and scaled to a range [−1, 1] to provide measured states for use in the next calculation step. FIG. 17 depicts the time evolution of four droplet states of the array during one run of the HCMC method implementing a stochastic gradient descent approach for solving the following problem Hamiltonian:

$${H}_{P} = {h}^{( 0 )} + {\sum }_{i = 1}^{4} {h}_{i}^{( 1 )} {s}_{i} + {\sum }_{i < j}^{4} {h}_{i j}^{( 2 )} {s}_{i} {s}_{j} , \qquad ( 23 )$$

$${h}^{( 0 )} = 1 , \qquad ( 24 )$$

$${h}^{( 1 )} = \frac{1}{4} [ \begin{matrix}0 & - 1 & - 2 & - 1\end{matrix} ] , \qquad ( 25 )$$

$${h}^{( 2 )} = \frac{1}{8} [ \begin{matrix}0 & 0 & 0 & - 2 \\ 0 & 0 & 1 & 0 \\ 0 & 1 & 0 & 1 \\ - 2 & 0 & 1 & 0\end{matrix} ] . \qquad ( 26 )$$

Mapping the final droplets states to a range [−1, 1] provided the following solution to the computational problem encoded in the problem Hamiltonian of Eq. (23): [+1 −1 +1 +1]. Referring to FIG. 18, there is illustrated a graph of the time evolution of the energy of the droplet system computed based on the droplet states depicted FIG. 17. FIG. 18 shows the system approaching its minimum energy and then stopping. It is appreciated that FIG. 18 depicts only the trajectory followed in one particular run and that different runs may have different trajectories.

### Model for a Polymerization-Based PMC Approach

A simple, idealized model for a PMC system based on polymerization reactions was studied. In the model, the state of a droplet is given by its average molecular weight (MW). Without loss of generality, a droplet with a low average MW (or high monomer concentration) is in state 0, and one with a high average MW (or low monomer concentration) is in state 1. The ability to drive and reverse polymerization of monomers is assumed to be achieved via an initiator and a depolymerizer, respectively.

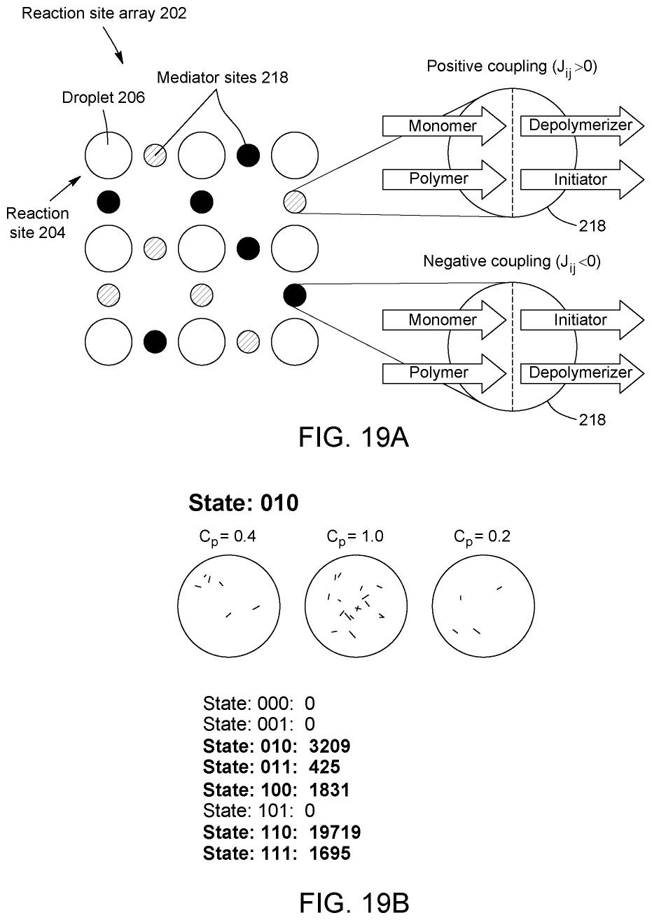

FIG. 19A illustrate a possible implementation of a model for such a polymerization-based PMC approach, with chemical mediator sites 218 governing the interaction between two neighboring reaction sites 204 containing droplets 206. For example, the chemical mediator sites 218 may be inspired by biological systems and contain smart sensing membranes with selective transporters. Upon being programmed according to the signs of the couplings between the droplets 206, the mediator sites 218 can control the release of initiator and depolymerizer molecules as a response to the MW distribution of their neighboring droplets 206 (simplified in FIG. 19A as “monomer” and “polymer” for low and high MWs, respectively). The initiator and depolymerizer molecules then diffuse to the droplets 206 and react with existing monomers and polymers, whose concentrations are determined through typical chemical kinetics. In FIG. 19A, the chemical mediator sites 218 are arranged in an array interlaced with the reaction sites array 202, but other arrangements are possible in other embodiments.

The model consists of the following reagents: monomer (M), polymer (P) consisting of a chain of monomers, initiator (I), and depolymerizer (D). The concentrations in droplets i are given by c_{M}ⁱ, c_{P}ⁱ, c_{I}ⁱ, and c_{D}ⁱ. Assuming all stoichiometric ratios are equal to one, the two reactions can be written as:

$$M + I {\to }^{{k}_{1}} P , \qquad ( 27 )$$

$$P + D {\to }^{{k}_{2}} M . \qquad ( 28 )$$

The resulting rate equations can be written as:

$$\frac{d [ P ]}{d t} = {k}_{1} [ I ] [ M ] - {k}_{2} [ D ] [ P ] . \qquad ( 29 )$$

$$\frac{d [ M ]}{d t} = - \frac{d [ P ]}{d t} , \qquad ( 30 )$$

Given the following Ising Hamiltonian, the ground state of the system can be reached by iterating over the following three steps.

$$H ( s ) = c + {\sum }_{i = 1}^{N} {h}_{i} {s}_{i} - {\sum }_{i < j}^{N} {J}_{i j} {s}_{i} {s}_{j} \qquad ( 31 )$$

Determination of the initiator (I) and depolymerizer (D) concentrations. The local field (one-body terms h, in the Ising Hamiltonian) at each droplet 206 corresponds to a constant on-site presence of initiator (I) and depolymerizer (D). In droplet i, the choice between I or D is given by the sign of hᵢ, and the concentration of either I or D is given by the magnitude of hᵢ, yielding:

$${h}_{i} \geq 0 \to \frac{d {c}_{D}^{i}}{d t} = {h}_{i} , \qquad ( 32 )$$

$${h}_{i} < 0 \to \frac{d {c}_{I}^{i}}{d t} = \vert {h}_{i} \vert . \qquad ( 33 )$$

The two-body terms Jᵢⱼ in the Ising Hamiltonian correspond to inter-droplet couplings. In FIG. 19A, the inter-droplet couplings are controlled by the chemical mediator sites 218. In the case of positive coupling (Jᵢⱼ>0), the chemical mediator site between droplets i and j is configured to respond to a low (high) average molecular weight at droplet i by increasing the concentration of depolymerizer (initiator) molecules at droplet j, and vice versa. In the case of negative coupling (Jᵢⱼ<0), the chemical mediator site between droplets i and j is configured to respond to a low (high) average molecular weight at droplet i by increasing the concentration of initiator (depolymerizer) molecules at droplet j, and vice versa.

The concentrations of I and D in droplet i depend on the strength of Jᵢⱼ and the concentration of M and P in neighboring droplet j, yielding:

$${J}_{i j} \geq 0 \to \frac{d {c}_{I}^{i}}{d t} = {c}_{M}^{j} {J}_{ij} , \frac{d {c}_{D}^{i}}{d t} = {c}_{P}^{j} {J}_{ij} , \qquad ( 35 )$$

$${J}_{i j} < 0 \to \frac{d {c}_{I}^{i} ,}{d t} = {c}_{P}^{j} \vert {J}_{i j} \vert , \frac{d {c}_{D}^{i}}{d t} = {c}_{M}^{j} \vert {J}_{i j} \vert . \qquad ( 36 )$$

It was found that the exact form of the response function, whether linear or nonlinear, generally does not change the ability of the simulation to reach a solution, as long as that the response function increases monotonically with concentration.

Update of the monomer (M) and polymer (P) concentrations. In each droplet 206, the M and P concentrations evolve following the kinetics of the chemical system. The rate laws can be written as:

$$\frac{d {c}_{P}^{i}}{d t} = {k}_{1} {c}_{I}^{i} {c}_{M}^{i} - {k}_{2} {c}_{D}^{i} {c}_{P}^{i} , \qquad ( 37 )$$

$$\frac{d {c}_{M}^{i}}{d t} = - \frac{d {c}_{P}^{i}}{d t} . \qquad ( 38 )$$

The state of droplet i is obtained from the normalized concentrations of M and P, as follows:

0<c_{P}<0.5→State 0, (39)

0.5<c_{P}<1→State 1. (40)

Resetting of the initiator (I) and depolymerizer (D) concentrations. Assuming that I and D are completely used up during the reaction (e.g. as in a RAFT agent): c_{I}ⁱ=0 and c_{D}ⁱ=0. By iterating steps 1 to 3, the potential energy surface is sampled, and the lowest-energy states can be located, corresponding to the most-probable states visited. For example, given the three-droplet 2-SAT problem: (s₁ or s₂) and (s₂ or not s₃), the corresponding Ising Hamiltonian to minimize is as follows:

$${H}_{P} = c + {\sum }_{i = 1}^{3} {h}_{i} {s}_{i} + {\sum }_{i < j}^{3} {J}_{i j} {s}_{i} {s}_{j} , \qquad ( 41 )$$

$$c = 2 , {h}_{i} = [ \begin{matrix}- 1 \\ - 2 \\ 1\end{matrix} ] , {J}_{i j} = [ \begin{matrix}0 & 1 & 0 \\ 1 & 0 & - 1 \\ 0 & - 1 & 0\end{matrix} ] . \qquad ( 42 )$$

FIG. 19B gives a snapshot of an HCMC-based simulation of the model after over 25,000 steps. The simulation can identify all the states that satisfy the 2-SAT problem (CNF above), which depicted in bold in FIG. 19B. It is appreciated that the simulation performed in this study may be generalized to any suitable physical system of the simplified form:

$$A + X {\to }^{{k}_{1}} B , \qquad ( 43 )$$

$$B + Y {\to }^{{k}_{1}} A . \qquad ( 44 )$$

### Problem Scaling

A factor to consider when a solving computational problem with an array of reaction sites (e.g., droplets) is how the number of reaction sites in the array scales with the number of problem variables in the problem Hamiltonian used to represent the computation problem. It is appreciated that compared to quantum annealers based on Josephson junctions operated at near absolute zero temperature, the cost of adding droplets to a molecular computer is generally negligible relative to the overall cost of the system. Furthermore, the parallelization of chemical reactions across all droplets may give molecular computers an advantage over numerical gradient evaluations in a classical computers.

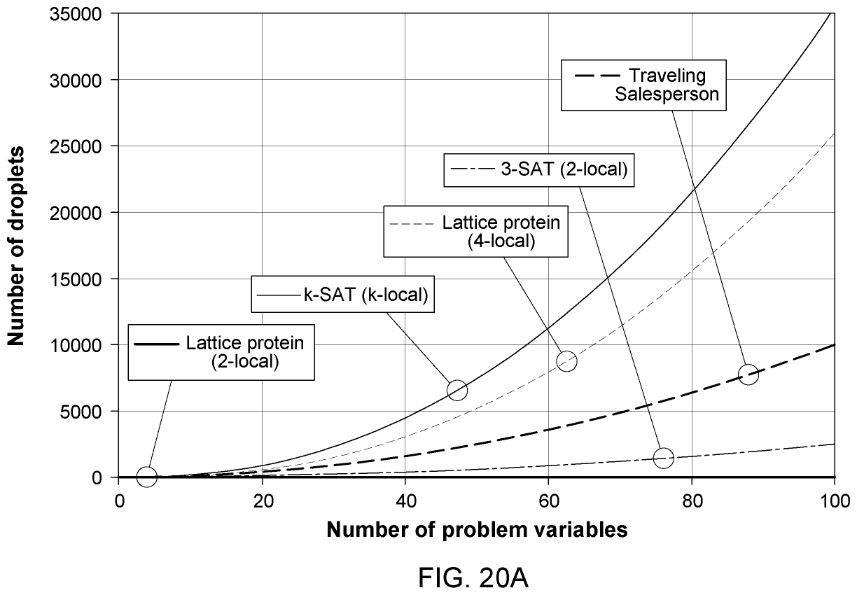

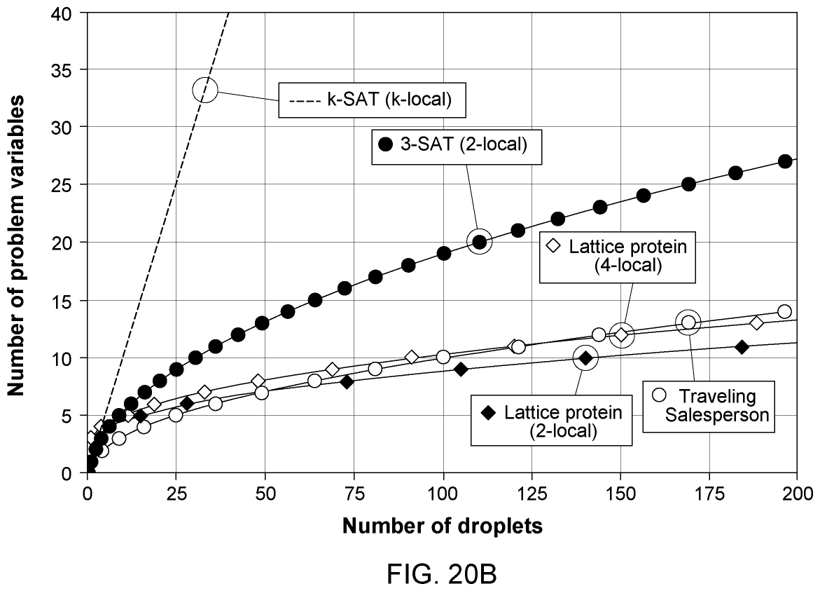

FIGS. 20A and 20B illustrate the scaling behavior of five different computational problems that can be solved using either or both of the molecular computing approaches disclosed herein: lattice protein folding problem (two-local and four-local); Boolean satisfiability problem (3-SAT, two-local and k-SAT, k-local); and traveling salesperson problem. The curves in FIG. 20A depict the scaling of the number of droplets for solving the five computational problems encoded with an Ising Hamiltonian, plotted as functions of the number of problem variables, while the curves in FIG. 20B depict the number of problem variables that the five computational problems can include, plotted as functions of the number of droplets in the array. As noted above, the HCMC approach may be fully connected and allows for k-local inter-droplet couplings, whereas the PMC approach is generally limited to two-local, nearest-neighbor inter-droplet couplings. Scaling relationships between the number of droplets and the number of problem variables can account for locality reduction algorithms and ancillary droplets.

In the HCMC approach, Boolean satisfiability for k variables per clause (k-SAT) scales linearly with the problem size, as O(n), where n is the number of problem variables. This is because, in this case, each problem variable corresponds to one droplet, and all droplets are connected to each other via control hardware. For three or more clauses, k≥3, the computational problem is NP-complete. In the PMC approach, reducing a 3-SAT problem to a two-local Ising Hamiltonian involves the use of (n−1)²/4 ancilla droplets. For the traveling salesperson problem involving n points or nodes, n² droplets are needed to label the sequence of points visited, thus the number of droplets scales as O(n²). Since the Ising Hamiltonian for the traveling salesperson problem includes only quadratic terms (two-local), it can be implemented in the PMC approach without locality reduction. For the lattice protein folding problem, in order to find the minimum-energy two-dimensional structure for a string of n amino acids, the four-local Hamiltonian encodes interaction energies between neighboring amino acids and self-avoidance, and the number of droplets scales as n² log(n). Furthermore, it has been shown that embedding QUBO problems on a sparsely graph scales linearly with the number of problem variables.

The time to solution (TTS) is a figure of merit (or performance metric) which considers the average time for one run and the average number of runs required to solve a problem. The TTS depends on the technology and performance of the computer/device and typically scales with the size of the problem (e.g., the number of problem variables).

As the number of droplets increases with the number of problem variables, so does generally the expectation that errors may accumulate as a result of noise, control, and readout. In order to reduce or help reduce such scalability-related issues, error correction schemes may be implemented. In some embodiments, classical bit repetition schemes may be used. In other embodiments, quantum annealing correction schemes may be adapted for use with the present techniques. On such scheme is the nested quantum annealing correction (NQAC) scheme, which involves making C copies of each bit and increasing the couplings by C². In the NQAC scheme, once calculation has been performed and the bits have been read, a majority vote is taken over the C copies of each bit to determine to its final state. It is appreciated that other types of error correction schemes may be used in other embodiments It is also appreciated that while error correction schemes may ensure or help ensure a higher probability of reaching a ground state solution, they also lead to an increase in the number of bits, or droplets, in the molecular computer.

Numerous modifications could be made to the embodiments described above without departing from the scope of the appended claims. In particular, while several embodiments have been shown and described, modifications and substitutions may be made thereto without departing from the scope of the present disclosure. Accordingly, it is to be understood that the present techniques have been described by way of illustrations and not limitation. Embodiments herein can be used independently or can be combined, unless stated otherwise.

1. A molecular computer system for solving a computational problem represented as a problem Hamiltonian expressed in terms of a set of problem variables and a set of problem parameters, the molecular computer system comprising:
an array of reaction sites having a physicochemical property that maps to at least two possible site states, each reaction site representing one of the problem variables, the reaction sites having site couplings enforced thereon that represent the problem parameters,
wherein, in one or more runs, the array is allowed to evolve, starting from an initial array configuration and subjected to the enforced site couplings, toward a final array configuration in which each reaction site is in one of the at least two possible site states, the final array configuration providing a solution to the computational problem.

2. The molecular computer system of claim 1, wherein the problem variables are binary variables, and wherein the at least two possible site states of the physicochemical property are a pair of possible site states.

3. The molecular computer system of claim 1, wherein the problem variables are n-valued variables, and wherein the at least two possible site states are a set of n possible site states, wherein n is a positive integer greater than two.

4. The molecular computer system of any one of claims 1 to 3, wherein the number of reaction sites is equal to the number of problem variables.

5. The molecular computer system of any one of claims 1 to 3, wherein the number of reaction sites is greater than the number of problem variables.

6. The molecular computer system of any one of claims 1 to 5, wherein the site couplings comprise inter-site couplings.

7. The molecular computer system of claim 6, wherein the site couplings further comprise intra-site couplings.

8. The molecular computer system of claim 7, further comprising intra-site couplers and inter-site couplers configured to enforce the intra-site couplings and the inter-site couplings by mass exchange, respectively.

9. The molecular computer system of claim 8, wherein the inter-site couplers are configured to operate by passive diffusion or electrokinetic transport.

10. The molecular computer system of claim 7, further comprising intra-site couplers and inter-site couplers configured to enforce the intra-site couplings and the inter-site couplings by energy exchange, respectively.

11. The molecular computer system of claim 10, wherein the intra-site couplers and the inter-site couplers are part of an electrode array.

12. The molecular computer system of any one of claims 6 to 11, wherein the problem parameters comprise two-body interaction parameters encoded in the inter-site couplings.

13. The molecular computer system of claim 12, wherein the problem Hamiltonian is expressed in an Ising representation or a quadratic unconstrained binary optimization (QUBO) representation.

14. The molecular computer system of any one of claims 6 to 11, wherein the problem parameters comprise k-body interaction parameters encoded in the inter-site couplings, wherein k is a positive integer greater than two.

15. The molecular computer system of claim 14, wherein the problem Hamiltonian is expressed in a polynomial unconstrained binary optimization (PUBO) representation.

16. The molecular computer system of any one of claims 1 to 15, wherein the computational problem is a combinatorial optimization problem.

17. The molecular computer system of claim 16, wherein the combinatorial optimization problem is an NP or NP-hard optimization problem.

18. The molecular computer system of claim 17, wherein the NP or NP-hard optimization problem is an NP-complete optimization problem.

19. The molecular computer system of any one of claims 1 to 18, wherein the array of reaction sites comprises an array of droplets.

20. The molecular computer system of claim 19, wherein the droplets each have a droplet volume ranging from about one nanoliter to about ten microliters.

21. The molecular computer system of claim 19 or 20, wherein the array of reaction sites comprises an array of containers, and the array of droplets is received in the array of containers.

22. The molecular computer system of claim 19 or 20, wherein the array of reaction sites comprises a non-wetting substrate, and the array of droplets is disposed on the non-wetting substrate.

23. The molecular computer system of claim 19 or 20, wherein the array of reaction sites comprises a gel substrate, and wherein the array of droplets is embedded in the gel substrate.

24. The molecular computer system of claim 19 or 20, wherein the array of reaction sites comprises a printing substrate, and wherein the array of droplets is printed on the printing substrate.

25. The molecular computer system of claim 24, wherein the printing substrate is made of paper or polymer.

26. The molecular computer system of any one of claims 1 to 25, wherein the array of reaction sites is arranged in a square lattice configuration or in a hexagonal lattice configuration.

27. The molecular computer system of any one of claims 1 to 26, wherein the physicochemical property comprises pH, polymer molecular weight, concentration, oxidation state, color, viscosity, a chemical oscillation property, or a combination thereof.

28. The molecular computer system of any one of claims 1 to 25, further comprising a site-readout device configured to read out the site states of the reaction sites in the final array configuration.

29. The molecular computer system of claim 28, wherein the site-readout device is configured to operate according to an optical readout scheme, an electrical readout scheme, an electrochemical readout scheme, or a combination thereof.

30. The molecular computer system of any one of claims 1 to 29, further comprising a site-manipulation device configured to prepare the array of reaction sites in the initial array configuration.

31. The molecular computer system of claim 30, wherein the site-manipulation device is configured to operate according to an optical actuation scheme, an electrical actuation scheme, an electrochemical actuation scheme, or a combination thereof.

32. The molecular computer system of any one of claims 1 to 31, wherein, in the initial array configuration, each reaction site is in one of the at least two possible site states.

33. The molecular computer system of any one of claims 1 to 32, wherein the solution to the computational problem corresponds to a ground state of the problem Hamiltonian.

34. The molecular computer system of any one of claims 1 to 32, wherein the solution to the computational problem comprises a plurality of solutions.

35. A molecular computer method for solving a computational problem represented as a problem Hamiltonian expressed in terms of a set of problem variables and a set of problem parameters, the molecular computer method comprising:
providing an array of reaction sites having a physicochemical property that maps to at least two possible site states, each reaction site representing one of the problem variables, the reaction sites having site couplings enforced thereon that represent the problem parameters;
performing, until an end condition has been met, one or more runs toward a solution to the computational problem, each run comprising:
allowing the array to evolve, starting from an initial array configuration and subjected to the enforced site couplings, to a final array configuration in which each reaction site is in one of the at least two possible site states;
reading out the site states of the reaction sites in the final array configuration;
determining a candidate solution to the computational problem from the read-out site states; and
determining whether the end condition has been met; and
determining the solution to the computational problem from at least one of the one or more candidate solutions.

36. The molecular computer method of claim 35, wherein the problem variables are binary variables, and wherein the at least two possible site states of the physicochemical property are a pair of possible site states.

37. The molecular computer method of claim 35, wherein the problem variables are n-valued variables, and wherein the at least two possible site states are a set of n possible site states, wherein n is a positive integer greater than two.

38. The molecular computer method of any one of claims 35 to 37, wherein providing the array of reaction sites comprises setting the number of reaction sites to be equal to the number of problem variables.

39. The molecular computer method of any one of claims 35 to 37, wherein providing the array of reaction sites comprises setting the number of reaction sites to be greater than the number of problem variables.

40. The molecular computer system of any one of claims 35 to 39, wherein the site couplings comprise inter-site couplings.

41. The molecular computer system of claim 40, wherein the site couplings further comprise intra-site couplings.

42. The molecular computer method of claim 41, wherein providing the array of reaction sites comprises enforcing the intra-site and the inter-site couplings by mass exchange.

43. The molecular computer method of claim 41, wherein providing the array of reaction sites comprises enforcing the intra-site and the inter-site couplings by energy exchange.

44. The molecular computer method of any one of claims 40 to 43, wherein the problem parameters comprise two-body interaction parameters encoded in the inter-site couplings.

45. The molecular computer method of claim 44, wherein the problem Hamiltonian is expressed in an Ising representation or a quadratic unconstrained binary optimization (QUBO) representation.

46. The molecular computer method of any one of claims 40 to 43, wherein the problem parameters comprise k-body interaction parameters encoded in the inter-site couplings, wherein k is a positive integer greater than two.

47. The molecular computer method of claim 46, wherein the problem Hamiltonian is expressed in a polynomial unconstrained binary optimization (PUBO) representation.

48. The molecular computer method of claim 46, further comprising performing a locality reduction process to reduce the k-body interaction parameters into two-body interaction parameters.

49. The molecular computer method of any one of claims 35 to 48, wherein the computational problem is a combinatorial optimization problem.

50. The molecular computer method of claim 49, wherein the combinatorial optimization problem is an NP or NP-hard optimization problem.

51. The molecular computer method of claim 50, wherein the NP or NP-hard optimization problem is an NP-complete optimization problem.

52. The molecular computer method of any one of claims 35 to 51, wherein the array of reaction sites comprises an array of droplets.

53. The molecular computer method of claim 52, wherein providing the array of reaction sites comprises disposing the array of droplets in an array of containers; or disposing the array of droplets on a non-wetting substrate; or embedding the array of droplets in a gel substrate; or printing the array of droplets on a printed substrate.

54. The molecular computer method of any one of claims 35 to 53, further comprising selecting the physicochemical property from pH, polymer molecular weight, concentration, oxidation state, color, viscosity, a chemical oscillation property, or a combination thereof.

55. The molecular computer method of any one of claims 35 to 54, wherein reading out the site states of the reaction sites in the final array configuration comprises operating an optical readout scheme, an electrical readout scheme, an electrochemical readout scheme, or a combination thereof.

56. The molecular computer method of any one of claims 35 to 55, further comprising preparing the array of reaction sites in the initial array configuration.

57. The molecular computer method of claim 56, wherein preparing the array of reaction sites in the initial array configuration comprises operating an optical actuation scheme, an electrical actuation scheme, an electrochemical actuation scheme, or a combination thereof.

58. The molecular computer method of any one of claims 35 to 57, wherein, in the initial array configuration, each reaction site is in one of the at least two possible site states.

59. The molecular computer method of any one of claims 35 to 58, wherein the one or more runs consist of a single run.

60. The molecular computer method of any one of claims 35 to 58, wherein the one or more runs consist of multiple runs.

61. The molecular computer method of claim 60, wherein the solution to the computational problem comprises a plurality of solutions.

62. The molecular computer method of any one of claims 35 to 51, wherein determining whether the end condition has been met comprises, for each run, assessing whether a specified number of run or runs has been completed, or assessing whether a specified allowed computation time has been reached, or assessing whether the candidate solution meets a specified solution criterion, or any combination thereof.

63. The molecular computer method of any one of claims 35 to 62, wherein the solution to the computational problem corresponds to a ground state of the problem Hamiltonian.

64. A hybrid classical-molecular computer (HCMC) system for solving a computational problem represented as a problem Hamiltonian expressed in terms of a set of problem variables and a set of problem parameters, the HCMC system comprising:
an array of reaction sites having a physicochemical property that maps to at least two possible site states, each reaction site representing one of the problem variables;
a digital computer operatively coupled to the array of reaction sites, the digital computer comprising a processor and a non-transitory computer readable storage medium having stored thereon computer readable instructions that, when executed by the processor, cause the processor to perform a method comprising:
performing one or more iterative cycles toward a solution to the computational problem, each iterative cycle comprising:
receiving, from a site-readout device, the site states of the reaction sites read out in an initial array configuration;
determining a candidate solution to the computational problem from the initial array configuration by inputting the read-out site states for the problem variables in the problem Hamiltonian;
determining whether an end condition has been met;
if the end condition has been met, terminating the one or more iterative cycles; and
if the end condition has not been met: determining, based on the problem parameters, state perturbations to be applied to a number of the reaction sites to promote state changes therein; controlling a site-manipulation device to apply the state perturbations; allowing the array to evolve, from the initial array configuration and responsive to the applied state perturbations, to a final array configuration; and setting the final array configuration as the initial array configuration for the next iterative cycle; and
determining the solution to the computational problem from at least one of the one or more candidate solutions.

65. The HCMC system of claim 64, wherein the problem variables are binary variables, and wherein the at least two possible site states of the physicochemical property are a pair of possible site states.

66. The HCMC system of claim 64, wherein the problem variables are n-valued variables, and wherein the at least two possible site states are a set of n possible site states, wherein n is a positive integer greater than two.

67. The HCMC system of any one of claims 64 to 66, wherein the number of reaction sites is equal to the number of problem variables.

68. The HCMC system of any one of claims 64 to 66, wherein the number of reaction sites is greater than the number of problem variables.

69. The HCMC system of any one of claims 64 to 68, wherein the problem parameters comprise two-body interaction parameters.

70. The HCMC system of claim 69, wherein the problem Hamiltonian is expressed in an Ising representation or a quadratic unconstrained binary optimization (QUBO) representation.

71. The HCMC system of any one of claims 64 to 70, wherein the problem parameters comprise k-body interaction parameters, wherein k is a positive integer greater than two.

72. The HCMC system of claim 71, wherein the problem Hamiltonian is expressed in a polynomial unconstrained binary optimization (PUBO) representation.

73. The HCMC system of any one of claims 64 to 72, wherein the computational problem is a combinatorial optimization problem.

74. The HCMC system of claim 73, wherein the combinatorial optimization problem is an NP or NP-hard optimization problem.

75. The HCMC system of claim 74, wherein the NP or NP-hard optimization problem is an NP-complete optimization problem.

76. The HCMC system of any one of claims 64 to 75, wherein the array of reaction sites comprises an array of droplets.

77. The HCMC system of claim 76, wherein the droplets each have a droplet volume ranging from about one nanoliter to about ten microliters.

78. The HCMC system of claim 76 or 77, wherein the array of reaction sites comprises an array of containers, and the array of droplets is received in the array of containers.

79. The HCMC system of claim 76 or 77, wherein the array of reaction sites comprises a non-wetting substrate, and the array of droplets is disposed on the non-wetting substrate.

80. The HCMC system of claim 76 or 77, wherein the array of reaction sites comprises a gel substrate, and wherein the array of droplets is embedded in the gel substrate.

81. The HCMC system of any one of claims 64 to 80, wherein the array of reaction sites is arranged in a square lattice configuration or in a hexagonal lattice configuration.

82. The HCMC system of any one of claims 64 to 81, wherein the physicochemical property comprises pH, polymer molecular weight, concentration, oxidation state, color, viscosity, a chemical oscillation property, or a combination thereof.

83. The HCMC system of any one of claims 64 to 82, wherein the site-readout device is configured to read out the site states of the reaction sites in the initial array configuration by operating an optical readout scheme, an electrical readout scheme, an electrochemical readout scheme, or a combination thereof.

84. The HCMC system of any one of claims 64 to 83, wherein the site-manipulation device is configured to apply the state perturbations to the number of reaction sites by operating an optical actuation scheme, an electrical actuation scheme, an electrochemical actuation scheme, or a combination thereof.

85. The HCMC system of any one of claims 64 to 84, wherein the solution to the computational problem corresponds to a ground state of the problem Hamiltonian.

86. The HCMC system of any one of claims 64 to 84, wherein the solution to the computational problem comprises a plurality of solutions.

87. The HCMC system of any one of claims 64 to 86, wherein, for each iterative cycle, the processor is configured for determining whether the end condition has been met by assessing whether a specified number of iterative cycles has been completed, or assessing whether a specified allowed computation time has been reached, or assessing whether the candidate solution meets a specified solution criterion, or any combination thereof.

88. The HCMC system of any one of claims 64 to 87, wherein the processor is configured for determining the state perturbations by performing a computational optimization operation.

89. The HCMC system of claim 88, wherein the computational optimization operation comprises a simulated annealing operation or stochastic gradient descent operation.

90. The HCMC system of any one of claims 64 to 89, wherein the processor is configured for: performing the one or more iterative cycles for a plurality of computational runs, each computational run having one or more candidate solutions associated therewith that define a respective one of a plurality of single-run solutions; and
determining the solution to the computational problem from the plurality of single-run solutions.

91. A hybrid classical-molecular computer (HCMC) method for solving a computational problem represented as a problem Hamiltonian expressed in terms of a set of problem variables and a set of problem parameters, the hybrid classical-molecular method comprising:
providing an array of reaction sites having a physicochemical property that maps to at least two possible site states, each reaction site representing one of the problem variables;
performing one or more iterative cycles toward a solution to the computational problem, each iterative cycle comprising:
reading out the site states of the reaction sites with the array in an initial array configuration;
determining a candidate solution to the computational problem from the initial array configuration by inputting the read-out site states for the problem variables in the problem Hamiltonian;
determining whether an end condition has been met;
if the end condition has been met, terminating the one or more iterative cycles; and
if the end condition has not been met:
determining, based on the problem parameters, state perturbations to be applied to a number of the reaction sites to promote state changes therein;
applying the state perturbations;
allowing the array to evolve, from the initial array configuration and responsive to the applied state perturbations, to a final array configuration; and
setting the final array configuration as the initial array configuration for the next iterative cycle; and
determining the solution to the computational problem from at least one of the one or more candidate solutions.

92. The HCMC method of claim 91, wherein the problem variables are binary variables, and wherein the at least two possible site states of the physicochemical property are a pair of possible site states.

93. The HCMC method of claim 91, wherein the problem variables are n-valued variables, and wherein the at least two possible site states are a set of n possible site states, wherein n is a positive integer greater than two.

94. The HCMC method of any one of claims 91 to 93, wherein providing the array of reaction sites comprises setting the number of reaction sites to be equal to the number of problem variables.

95. The HCMC method of any one of claims 91 to 93, wherein providing the array of reaction sites comprises setting the number of reaction sites to greater than the number of problem variables.

96. The HCMC method of any one of claims 91 to 95, wherein the problem parameters comprise two-body interaction parameters.

97. The HCMC method of claim 96, wherein the problem Hamiltonian is expressed in an Ising representation or a quadratic unconstrained binary optimization (QUBO) representation.

98. The HCMC method of any one of claims 91 to 97, wherein the problem parameters comprise k-body interaction parameters, wherein k is a positive integer greater than two.

99. The HCMC method of claim 98, wherein the problem Hamiltonian is expressed in a polynomial unconstrained binary optimization (PUBO) representation.

100. The HCMC method of any one of claims 91 to 99, wherein the computational problem is a combinatorial optimization problem.

101. The HCMC method of claim 100, wherein the combinatorial optimization problem is an NP or NP-hard optimization problem.

102. The HCMC method of claim 101, wherein the NP or NP-hard optimization problem is an NP-complete optimization problem.

103. The HCMC method of any one of claims 91 to 102, wherein the array of reaction sites comprises an array of droplets.

104. The HCMC method of claim 103, wherein providing the array of reaction sites comprises disposing the array of droplets in an array of containers; or disposing the array of droplets on a non-wetting substrate; or embedding the array of droplets in a gel substrate; or printing the array of droplets on a printed substrate.

105. The HCMC method of any one of claims 91 to 104, further comprising selecting the physicochemical property from pH, polymer molecular weight, concentration, oxidation state, color, viscosity, a chemical oscillation property, or a combination thereof.

106. The HCMC method of any one of claims 91 to 105, wherein reading out the site states of the reaction sites in the initial array configuration comprising operating an optical readout scheme, an electrical readout scheme, an electrochemical readout scheme, or a combination thereof.

107. The HCMC method of any one of claims 91 to 106, wherein applying the state perturbations to the number of reaction sites comprises operating an optical actuation scheme, an electrical actuation scheme, an electrochemical actuation scheme, or a combination thereof.

108. The HCMC method of any one of claims 91 to 107, wherein the solution to the computational problem corresponds to a ground state of the problem Hamiltonian.

109. The HCMC method of any one of claims 91 to 108, wherein the solution to the computational problem comprises a plurality of solutions.

110. The HCMC method of any one of claims 91 to 109, wherein determining whether the end condition has been met comprises, for each iterative cycle, assessing whether a specified number of iterative cycles has been completed, or assessing whether a specified allowed computation time has been reached, or assessing whether the candidate solution meets a specified solution criterion, or any combination thereof.

111. The HCMC method of any one of claims 91 to 110, wherein determining the state perturbations comprises performing a computational optimization operation.

112. The HCMC method of claim 111, wherein the computational optimization operation comprises a simulated annealing operation or stochastic gradient descent operation.

113. The HCMC method of any one of claims 91 to 112, further comprising: performing the one or more iterative cycles for a plurality of computational runs, each computational run having one or more candidate solutions associated therewith that define a respective one of a plurality of single-run solutions; and determining the solution to the computational problem from the plurality of single-run solutions.

114. A non-transitory computer readable storage medium having stored thereon computer readable instructions that, when executed by a processor, cause the processor to perform a method for solving a computational problem using an array of reaction sites, the computational problem being represented as a problem Hamiltonian expressed in terms of a set of problem variables and a set of problem parameters, and the array of reaction sites having a physicochemical property that maps to at least two possible site states, each reaction site representing one of the problem variables, the method comprising:
performing, by the processor, one or more iterative cycles toward a solution to the computational problem, each iterative cycle comprising:
receiving the site states of the reaction sites read out in an initial array configuration;
determining a candidate solution to the computational problem from the initial array configuration by inputting the read-out site states for the problem variables in the problem Hamiltonian;
determining whether an end condition has been met;
if the end condition has been met, terminating the one or more iterative cycles; and
if the end condition has not been met:
determining, based on the problem parameters, state perturbations to be applied to a number of the reaction sites to promote state changes therein;
controlling a site-manipulation device to apply the state perturbations;
allowing the array to evolve, from the initial array configuration and responsive to the applied state perturbations, to a final array configuration; and
setting the final array configuration as the initial array configuration for the next iterative cycle; and
determining, by the processor, the solution to the computational problem from at least one of the one or more candidate solutions.

115. The non-transitory computer readable storage medium of claim 114, wherein the problem variables are binary variables, and wherein the at least two possible site states of the physicochemical property are a pair of possible site states.

116. The non-transitory computer readable storage medium of claim 114 or 115, wherein the problem parameters comprise two-body interaction parameters.

117. The non-transitory computer readable storage medium of any one of claims 114 to 116, wherein the problem parameters comprise k-body interaction parameters, wherein k is a positive integer greater than two.

118. The non-transitory computer readable storage medium of any one of claims 114 to 117, wherein the computational problem is a combinatorial optimization problem.

119. The non-transitory computer readable storage medium of claim 118, wherein the combinatorial optimization problem is an NP or NP-hard optimization problem.

120. The non-transitory computer readable storage medium of claim 119, wherein the NP or NP-hard optimization problem is an NP-complete optimization problem.

121. The non-transitory computer readable storage medium of any one of claims 114 to 120, wherein the solution to the computational problem corresponds to a ground state of the problem Hamiltonian.

122. The non-transitory computer readable storage medium of any one of claims 114 to 121, wherein the solution to the computational problem comprises a plurality of solutions.

123. The non-transitory computer readable storage medium of any one of claims 114 to 122, wherein determining whether the end condition has been met comprises, for each iterative cycle, assessing whether a specified number of iterative cycles has been completed, or assessing whether a specified allowed computation time has been reached, or assessing whether the candidate solution meets a specified solution criterion, or any combination thereof.

124. The non-transitory computer readable storage medium of any one of claims 114 to 123, wherein determining the state perturbations comprises performing a computational optimization operation.

125. The non-transitory computer readable storage medium of claim 124, wherein the computational optimization operation comprises a simulated annealing operation or stochastic gradient descent operation.

126. The non-transitory computer readable storage medium of any one of claims 114 to 125, wherein the method further comprises: performing the one or more iterative cycles for a plurality of computational runs, each computational run having one or more candidate solutions associated therewith that define a respective one of a plurality of single-run solutions; and determining the solution to the computational problem from the plurality of single-run solutions.

127. A digital computer comprising:
a processor; and
the non-transitory computer readable storage medium of any one of claims 114 to 126, the non-transitory computer readable storage medium being operatively coupled to the processor.
